(19) United States
(12) Patent Application Publication
(10) Pub. No.: US 2017/0138937 A1
(43) Pub. Date: May 18, 2017

(54) DETECTION OF ANALYTES

(71) Applicant: TGR Biosciences Pty Ltd, Thebarton (AU)

(72) Inventors: Antony James Sheehan, Seaview Downs (AU); Ronald Ian William Osmond, Henley Beach South (AU); Michael Francis Crouch, Hackney (AU); Anthony Ross Dyer, Parkside (AU)

(21) Appl. No.: 15/188,594

(22) Filed: Jun. 21, 2016

Related U.S. Application Data
(63) Continuation of application No. 14/008,430, filed on Dec. 18, 2013, now abandoned.
(60) Provisional application No. 61/470,359, filed on Mar. 31, 2011.
(60) Provisional application No. 61/470,395, filed on Mar. 31, 2011.

(51) Int. Cl. G01N 33/543

(52) U.S. Cl. CPC G01N 33/54306

(57) ABSTRACT

The present disclosure relates to a one-step immunoassay, in which a solid substrate is pre-coated with an immobilisation agent, and whereby the capture agent, the analyte and the detection agent are added to the solid substrate together, followed by a wash step prior to detection. Methods and kits for detecting an analyte in a sample are disclosed. The capture agent can bind the analyte and comprises a ligand for an immobilisation agent. Certain embodiments are directed to antibody capture agents and/or antibody detectable agents. Certain embodiments are directed to a ligand comprising a peptide tag and an immobilisation agent comprising an anti-peptide tag antibody. Certain embodiments are directed to detection of more than one analyte.

### PRIORITY

This application claims priority from, and hereby incorporates by reference in their entirety, each of the following applications: U.S. provisional patent application 61/470,359 filed 31 Mar. 2011 entitled “Detection of Analytes” and U.S. provisional patent application 61/470,395 filed 31 Mar. 2011 entitled “Improved Immunoassay”.

### FIELD

The present disclosure relates to methods and/or kits for detecting an analyte in a sample. In some embodiments, the present disclosure relates to detecting an analyte using an antibody capture agent and/or an antibody detectable agent.

### BACKGROUND

Detection of analytes in samples is important in many industries including, for example, research, immunology, water quality assessment, environmental science and engineering, and medicine.

Some methods for detecting analytes rely on intrinsic properties of the molecule to be detected. For example, methods for detecting analytes in samples such as chemical analysis, high pressure liquid chromatography and mass spectrometry rely on chemical or physical properties of the molecules being detected (for example reactive groups, charge, size, and hydrophobicity).

Other methods for detecting analytes rely on the some part of the analyte being recognised by another molecule. For example, methods for detecting nucleic acids often utilise binding between nucleic acids having complementary base pairing. Other methods rely on the conformation of part of the analyte being recognised by another molecule. Examples of such methods of detection include an antibody binding to an antigen and an aptamer binding to a target region.

Methods that utilise recognition of an analyte by another molecule for detection are important, particularly in the life sciences. Generally such methods require extensive handling and washing before the analyte can be detected in a sample. For example, immunoassays utilising an antibody to detect an analyte through binding to an antigenic region of the analyte require a number of handling and washing steps before an analyte can be detected in the sample. In addition, such assays often suffer from a number of other deficiencies, such as the time that is required to ensure that the assay performs reliably and can also suffer variability depending upon the sample type.

For example, while enzyme-linked immunosorbent assays (ELISA) are a relatively common and inexpensive detection method, it generally includes at least 2 separate incubation and washing steps, and takes over 2 hours to complete. These deficiencies are particularly apparent when an antibody is used to capture the analyte on a solid substrate as part of the method of detection.

Accordingly, it would be desirable to provide a method and/or kits for detecting an analyte in a sample to address one or more other problems in the art and/or provide one or more advantages.

Reference to any prior art in this specification is not, and should not be taken as, an acknowledgment or any form of suggestion that this prior art forms part of the common general knowledge in any country.

### SUMMARY

The present disclosure relates to methods and/or kits for detecting an analyte in a sample.

In certain embodiments, the present disclosure provides a method for detecting an analyte in a sample, the method comprising:
providing a reaction vessel;
providing a solid substrate comprising a bound immobilisation agent;
providing a capture agent which can bind the analyte, wherein the capture agent comprises a ligand for the immobilisation agent;
providing a detectable agent which can bind to the analyte;
contacting the sample, the capture agent, the detectable agent and the solid substrate in the reaction vessel;
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

In certain embodiments, the present disclosure provides a method for detecting a non-nucleic acid analyte in a sample, the method comprising:
providing a reaction vessel;
providing a solid substrate comprising a bound immobilisation agent;
providing an antibody capture agent in solution which can bind the analyte, wherein the capture agent comprises a ligand for the immobilisation agent;
providing a detectable agent in solution which can bind to the analyte;
contacting the sample, the capture agent, the detectable agent and the solid substrate in the reaction vessel;
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

In certain embodiments, the present disclosure provides a kit for detecting an analyte, the kit comprising:
an assay platform comprising a plurality of reaction vessels, one or more of the reaction vessels comprising a bound immobilisation agent;
a capture agent which can bind to the analyte, wherein the capture agent comprises a ligand for the immobilisation agent;
a detectable agent which can bind to the analyte; and
optionally one or more solutions for washing the solid substrate and/or instructions for detecting the analyte.

In certain embodiments, the present disclosure provides a method for detecting one or more analytes in one or more samples using a single assay platform, the method comprising:
providing one or more samples comprising one or more analytes to be detected;
providing an assay platform comprising a plurality of reaction vessels, one or more of the plurality of reaction vessels comprising the same bound immobilisation agent;
providing one or more capture agents, the one or more capture agents being able to bind to the one or more analytes to be detected and comprising a ligand for the immobilisation agent;
providing one or more detectable agents, the one or more detectable agents being able to bind to the one or more analytes to be detected;
contacting the one or more samples, the one or more capture agents and the one or more detectable agents in one or more of the plurality of reaction vessels in the assay platform;
washing one or more of the plurality of reaction vessels to remove the one or more capture agents and the one or more detectable agents not bound via the ligand in one or more of the plurality of reaction vessels; and
detecting the one or more analytes in one or more of the plurality of reaction vessels by detecting the presence of the one or more detectable agents bound to one or more of the plurality of reaction vessels.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows for the purposes of comparison, three ELISA protocols for the detection of phosphorylated ERK 1/2 (pERK) were examined, using various concentrations of a cellular lysate containing pERK. (1) A simultaneous ELISA format, whereby the assay components, namely the capture antibody (anti-pERK-biotin), the analyte (cellular lysate), and the detection antibody (anti-ERK-HRP), were incubated concurrently in a streptavidin-coated microplate. (2) A standard multi-incubation ELISA format, whereby the capture antibody was first incubated in a streptavidin-coated microplate, followed by the analyte, and finally the detection antibody. (3) A standard multi-incubation ELISA format, whereby the analyte was incubated in a capture-antibody coated microplate, followed by a detection antibody. The assays were incubated for either 30 min (FIG. 1A) for each incubation step or 60 min (FIG. 1B) for each incubation step, and the wells were subjected to a standard wash cycle between each incubation step for each assay. After the final incubation and wash, QuantaRed™ HRP substrate was added to the wells, and each plate was incubated for 10 min in the dark. The fluorescent signal in the wells was measured at 550ex/600em nm. FIGS. 1A and 1B show the mean and standard deviations for the duplicate data points at each pERK lysate concentration analyzed. In this Figure the comparison clearly demonstrate comparable assay performance over a shorter time period when the assay components are incubated concurrently, compared with standard ELISA protocols whereby assay components are incubated sequentially.

FIG. 2 shows for the purposes of comparison, three ELISA protocols for the detection of phosphorylated ERK 1/2 (pERK), using various concentrations of a cellular lysate containing pERK. (1) A simultaneous ELISA format, whereby the assay components, namely the capture antibody (anti-pERK-biotin), the analyte (cellular lysate), and the detection antibody (anti-ERK-HRP), were incubated concurrently in a streptavidin-coated microplate for either 30 min (FIG. 2A) or 60 min (FIG. 2B). (2) A standard multi-incubation ELISA format, whereby the capture antibody was first incubated in a streptavidin-coated microplate for 10 min, followed by the analyte for 10 min, and finally the detection antibody for 10 min, giving a total cumulative assay incubation time of 30 min (FIG. 2A). (3) A standard multi-incubation ELISA format, whereby the analyte was incubated in a capture-antibody coated microplate for 30 min, followed by the detection antibody for 30 min, giving a total cumulative assay time of 60 min (FIG. 1B). The wells were subjected to a standard wash cycle between each incubation step for each assay. After the final incubation and wash, QuantaRed™ HRP substrate was added to the wells, and each plate was incubated for 10 min in the dark. The fluorescent signal in the wells was measured at 550ex/600em nm. FIGS. 2A and 2B show the mean and standard deviations for the duplicate data points at each pERK lysate concentration analyzed. In this Figure, the comparison clearly demonstrate better assay performance the same total assay time period when the assay components are incubated concurrently, compared with standard ELISA protocols whereby assay components are incubated sequentially.

FIG. 3 shows for the purposes of comparison, the concentration of the capture antibody (anti-phospho-ERK) required for optimal assay performance for three ELISA protocols for the detection of phosphorylated ERK 1/2, using varying concentrations of the capture antibody in combination with a fixed concentration of both cellular lysate, and detection antibody. (1) A simultaneous ELISA format, whereby the assay components, namely the capture antibody (anti-pERK-biotin), the analyte (cellular lysate), and the detection antibody (anti-ERK-HRP), were incubated concurrently in a streptavidin-coated microplate for 120 min. (2) A simultaneous ELISA format, whereby the assay components, namely the capture antibody (anti-pERK-peptide), the analyte (cellular lysate), and the detection antibody (anti-ERK-HRP), were incubated concurrently in an anti-peptide antibody-coated microplate for 120 min. (3) A standard multi-incubation ELISA format, whereby the analyte was incubated in a capture-antibody (non-biotinylated) coated microplate for 120 min, followed by the detection antibody for 120 min. The wells were subjected to a standard wash cycle between each incubation step for each assay. After the wash cycle, HRP substrate was added to the wells, and the plates were incubated for 10 min in the dark. The fluorescent signal in the wells was measured at 540ex/590em nm. FIG. 3 shows the mean and standard deviations for the duplicate data points for each target analyzed. In this Figure, the comparison clearly demonstrates that optimal assay performance is achieved with lower capture antibody concentrations when the assay components are incubated concurrently for both biotin-capture and peptide-capture protocols, when compared with standard ELISA protocols whereby analytes are incubated sequentially, and washed between incubations. This data demonstrates that the assay has the potential to lower input costs for ELISA plate manufacture.

FIG. 4 shows for the purposes of comparison, the requirement for sequential incubations for optimal assay performance for two ELISA protocols for the detection of phosphorylated ERK 1/2. (1) A simultaneous ELISA format, whereby the assay components, namely the capture antibody (anti-phospho-ERK-peptide), the analyte (cellular lysate), and the detection antibody (anti-ERK-HRP), were incubated concurrently in an antipeptide antibody-coated microplate for 120 min. (2) A sequential ELISA format, whereby the solution-phase assay components, namely the capture antibody (anti-pERK-peptide), the analyte (cellular lysate), and the detection antibody (anti-ERK-HRP), were incubated concurrently in a separate reaction vessel for 60 min. The assay components were subsequently transferred to an antipeptide antibody-coated microplate for 60 min. At the conclusion of incubation on the antipeptide antibody-coated assay microplate, both protocols required a standard wash cycle. After the wash cycle, HRP substrate was added to the wells, and the plates were incubated for 10 min in the dark. The fluorescent signal in the wells was measured at 540ex/590em nm.

FIG. 4 shows the mean and standard deviations for the duplicate data points for each target analyzed. In this Figure, the comparison clearly demonstrates that no benefit to assay performance is achieved with the inclusion of a pre-incubation step prior to introduction to a solid substrate carrying the immobilization agent.

FIG. 5 shows a single-incubation, single-wash ELISA, was performed using a 3-antibody configuration. The assay components, namely the capture antibody (anti-pERK-biotin), the analyte (cellular lysate), the detection antibody (rabbit anti-ERK), and a generic anti-rabbit-HRP antibody, were incubated concurrently in a streptavidin-coated microplate for 120 min (signal), and compared with a similar assay run with a buffer-only control for the analyte (noise). The wells were subjected to a standard wash cycle after the incubation step, and SigmaFAST™ HRP substrate was added to the wells, and each plate was incubated for 10 min in the dark. The colorimetric signal in the wells was measured at 450 nm. FIG. 5 shows the mean and standard deviations for the duplicate data points at each pERK lysate concentration analyzed. In this Figure, the assay clearly demonstrates the utility whereby the assay components are incubated concurrently.

FIG. 6 shows the detection of different kinases by a single incubation, single wash ELISA. Cell lysates containing either phosphorylated S6 p240/44, AKT pT308 or AKT pS473 (signal), or buffer-only controls (noise) were added to separate wells of an assay microplate (streptavidin coated 384-well Nunc Maxisorp™ plate). The reaction was started by the addition of target-specific antibody pairs (one biotinylated and the other conjugated to HRP) to the lysates. The assays were incubated for 2 h, then subjected to a wash cycle. After the wash cycle, QuantaRed™ HRP substrate was added to the wells, and the plate was incubated for 10 min in the dark. The fluorescent signal in the wells was measured at 550ex/600em nm. FIG. 6 shows the mean and standard deviations for the duplicate data points for each target analyzed. In this Figure, the assay clearly demonstrates efficacy for several different targets, whereby the assay components are incubated concurrently.

FIG. 7 is a schematic diagram showing a microfluidic cartridge suitable for use in accordance with some embodiments of the present disclosure.

FIG. 8 demonstrates the results of electrochemical detection of pERK in a microfluidic system. FIG. 8A shows the raw results of electrochemical detection (in mV) during the substrate flow through phase and substrate incubation phase. FIG. 8B shows a pERK standard curve generated using data taken from 60 seconds after injection of substrate (during flow through phase). FIG. 8C shows a pERK standard curve generated using data taken from 180 seconds after injection of substrate (at the end of the substrate incubation phase).

FIG. 9 shows the results of electrochemical detection of pAKT473 in a microfluidic system. FIG. 9A shows the raw results of electrochemical detection (in mV) during the substrate flow through phase and substrate incubation phase. FIG. 9B shows a pAKT473 standard curve generated using data taken from 60 seconds after injection of substrate (during flow through phase). FIG. 9C shows a pAKT473 standard curve generated using data taken from 180 seconds after injection of substrate (at the end of the substrate incubation phase).

FIG. 10 demonstrates equivalent assay performance with various permutations on the order of delivery of assay components to the assay well, using (A) a peptide capture antibody conjugate (anti-pERK-peptide), (B) or biotin capture antibody conjugate (anti-pERK-biotin), as the assay capture reagent The assay components were added in various permutations (refer to example 7, Tables 1 and 2). Individual assay components were added 1 min apart to the plates and incubated for 2 h at room temperature, then subjected to a wash cycle. After the wash cycle, HRP substrate was added to the wells, and the plates were incubated for 10 min in the dark. The fluorescent signal in the wells was measured at 540ex/590em nm. FIG. 10 shows the mean and standard deviations for the duplicate data points for each target analyzed. In this Figure, the assays clearly demonstrate that when added within the short time period described, the order of addition of individual assay components does not affect assay performance, compared with assay components that are added simultaneously.

FIG. 11 shows detection of recombinant human EGF, IL-2 and TNFα, in either PBS/0.5% BSA or human serum. Peptide-capture antibody conjugates, and HRP-detection antibody conjugates specific for each of EGF (A), IL-2 (B) and TNFα (C) were prepared. Recombinant EGF, IL-2 or TNFα were prepared at concentrations ranging from 100 ng/mL to 10 fg/mL, in either PBS/0.5% BSA, or human serum, and 50 μL/well of each analyte was added to an ELISA assay plate coated with an anti-peptide antibody. The assays were initiated by addition of mixtures containing both specific antibodies for each of EGF, IL-2 or TNFα, along with a general anti-HAMA composition available commercially from Bioreclamation LLS (Westbury, N.Y., USA—‘Immunoglobulin Inhibiting Reagent (IIR)), to the appropriate ELISA plate wells. The assays were incubated for 1 h, then subjected to a wash cycle. After the wash cycle, HRP substrate was added to the wells, and the plates were incubated for 10 min in the dark. The fluorescent signal in the wells was measured at 540ex/590em nm. FIG. 11 shows the mean and standard deviations for the duplicate data points for each target analyzed. In this Figure, the assay clearly demonstrates efficacy for several different targets in serum, whereby the assay components are incubated concurrently. The high signal for EGF in human serum is due to the presence of endogenous EGF protein(s) in this medium.

FIG. 12 shows detection of recombinant human EGF, IL-2 and TNFα in a 15 min total assay time. Peptide-capture antibody conjugates, and HRP-detection antibody conjugates specific for each of EGF (A), IL-2 (B) and TNFα (C) were prepared. Recombinant EGF, IL-2 or TNFα were prepared at concentrations ranging from 100 ng/mL to 10 fg/mL, in PBS/0.5% BSA, and 50 μL/well of each analyte was added to an ELISA assay plate coated with an anti-peptide antibody. The assays were initiated by addition of mixtures containing both specific antibodies for each of EGF, IL-2 or TNFα to the appropriate ELISA plate wells. The assays were incubated for 10 min, then subjected to a wash cycle. After the wash cycle, HRP substrate was added to the wells, and the plates were incubated for 5 min in the dark. The fluorescent signal in the wells was measured at 540ex/590em nm. FIG. 12 shows the mean and standard deviations for the duplicate data points for each target analyzed. In this Figure, the assay clearly demonstrates efficient detection within 15 min total assay time for several different targets, using certain embodiments, whereby the assay components are incubated concurrently.

FIG. 13 shows intra-plate variation observed for 2 separate single-incubation ELISAs for either phospho-AKT (pSer473) or phospho-STAT3.

FIG. 14 shows detection of TNFα in tissue culture supernates.

FIG. 15 shows detection of either phospho-AKT (pSer473) or phospho-ERK in a 25 min total assay time. For each target, recombinant active (A) phospho-AKT or (B) phospho-ERK was diluted as indicated, to various concentrations using 1× Lysis buffer containing 0.1% BSA and added to 4 replicate wells of a 96-well streptavidin-coated microplate. To initiate the assay reaction, for either target, a mixture of the biotin-conjugated capture antibody, and the HRP-conjugated detection antibody were added to the lysates, and incubated for 1 hour. The wells were subjected to a standard wash cycle for each assay. After the wash cycle, QuantaRed™ HRP substrate was added to the wells, and each plate was incubated for 10 min in the dark. The fluorescent signal in the wells was measured at 550ex/600em nm. FIGS. 15A and 15B show the data points at each analyte concentration analyzed, for phospho-AKT and phospho-ERK, respectively. Both assays demonstrated sensitivity to less than 1 ng/mL.

FIG. 16 shows detection of various concentrations of IL-2 using a peptide tag/anti-peptide tag antibody capture system.

FIG. 17 shows detection of various concentrations of IL-2 using a peptide tag anti peptide tag antibody capture system.

FIG. 18 shows detection of various concentrations of EGF, IL-2 & TNFα using a peptide tag anti peptide tag antibody capture system.

FIG. 19 shows the signal obtained for various concentrations of analyte using a peptide tag anti peptide tag antibody capture system.

FIG. 20 shows a comparison of a biotin-streptavidin capture system to a peptide tag-anti-peptide antibody capture system in various biological milieu.

FIG. 21A shows that a streptavidin biotin capture system utilizing an antibody capture agent and an antibody detectable agent is not affected by increasing concentrations of irrelevant antibodies. FIG. 21B shows the data from FIG. 20 has been normalised in terms of signal:noise, where noise is the signal of the immunocomplex obtained for each condition compared to the signal obtained in the absence of analyte.

FIG. 22A shows that anti peptide tag antibody-peptide capture system utilizing an antibody capture agent and an antibody detectable agent is not affected by increasing concentrations of irrelevant antibodies. FIG. 22B shows the data from FIG. 22A has been normalised in terms of signal:noise, where noise is the signal of the immunocomplex obtained for each condition compared to the signal obtained in the absence of analyte.

FIG. 23 shows the detection of lanthanide-labelled antibodies. Two antibodies were labelled with lanthanide with PerkinElmer labelling kits: An anti-EGF antibody was labelled with Europium using an Eu-labelling kit, PerkinElmer cat number 1244-302. An IL-2 antibody was labelled with Samarium using a Sm-labelling kit, PerkinElmer cat number 1244-303. Antibodies were incubated with the respective lanthanide solutions for 16 hours at room temperature, and conjugates were then desalted using a PD10 column. Standard curves for each conjugate were constructed using log 10 dilutions of the conjugates, maximal concentrations for Eu and Sm being 10 nM and 100 nM, respectively. The time-resolved fluorescence readings for these solutions were assessed in microtitre plates in a Victor II plate reader (PerkinElmer). Excitation and emission wavelengths for these lanthanides were: Europium: Excitation 340 nm/Emission 615 nm; Samarium: 340 nm/Emission 642 nm.

FIG. 24 shows the duoplexed detection of EGF and IL-2 using lanthanide-labelled antibodies in a plate reader using time-resolved fluorescence detection.

### DETAILED DESCRIPTION

The present disclosure relates to methods and/or kits for detecting an analyte in a sample.

Certain embodiments of the present disclosure provide methods and/or kits for detecting an analyte in a sample that have one or more combination of advantages. For example, some of the advantages of some embodiments of the methods and/or kits disclosed herein include: reducing the time taken to detect the analyte; reducing the number of step and/or duration for washing of the solid substrate; reducing the number of incubation steps; providing reliable performance; eliminating the need for pre-incubation; reducing the number of dispensing steps; reducing the number of aspiration steps; providing a simple easy to use assay; being suitable for microfluidic systems and/or other automated systems; reducing costs in materials; reducing costs in time needed to perform the assay; reducing handling costs; reducing handling errors; reducing the cost of the materials needed; measuring multiply analytes on one plate; being compatible for use with most standard plate readers; improving the ability to detect analytes in a variety of biological milieu; and/or providing improved sensitivity. Other advantages of certain embodiments of the present disclosure are also disclosed herein. For example, being able in some embodiments to tolerate samples with moderate to high levels of proteins; allowing the use of antibodies that have a low binding affinity to an analyte; allowing the use of lower concentration of antibodies to an analyte; and/or providing kits and/or assay platforms (such as microtitre plates) that are easy to manufacture and/or less costly to manufacturer. Other advantages are disclosed herein.

Certain embodiments of the present disclosure provide a method for detecting an analyte in a sample, the method comprising:
providing a reaction vessel;
providing a solid substrate comprising a bound immobilisation agent;
providing a capture agent which can bind the analyte, wherein the capture agent comprises a ligand for the immobilisation agent;
providing a detectable agent which can bind to the analyte;
contacting the sample, the capture agent, the detectable agent and/or the solid substrate in the reaction vessel;
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

Without being bound by theory, the present disclosure arises at least in part from the recognition that the underlying molecular events for detecting an analyte, by a process involving capture of the analyte on a solid substrate, may be affected by one or more of the way the analyte is captured, the handling and/or washing steps during the assay, and/or the timing and presentation of the various molecular species with each other or combinations thereof.

As a consequence, certain embodiments of the methods and/or kits of the present disclosure result in a number of advantages over previous assays. For example, the methods and/or kits of the present disclosure may in some embodiments result in improvement in the ability to detect an analyte in some types of samples and/or an improvement in the number of handling and/or washing steps required during an assay. In addition, the methods and/or kits of the present disclosure may in some embodiments result in an improvement in the time required to reliably detect the analyte.

Further, the improvements may in some embodiments provide a number of advantages over previous assays for detecting an analyte utilising an antibody (or an antigen binding part thereof) to capture an antigen. For example, the methods and/or kits of the present disclosure may in some embodiments result in an assay that can use a lower amount of one or more of capture agent, immobilisation agent and detectable agent, has a lower cost to perform and/or which provides more consistent results over previous assays.

By way of illustration, the ability to detect an analyte by ELISA can often be highly variable and dependent upon the sample type. The methods and/or kits of the present disclosure may in some embodiments provide an assay which is less variable and less affected by the sample type. For example, the use of capture agents utilising a peptide tag and an immobilised anti-peptide antibody may in some embodiments result in improved detection of an analyte in some sample types and/or assist in reducing the washing of the solid substrate.

ELISA can also take up to 6 hours to complete and consist of at least 2 separate incubation and washing steps. Other enzyme-linked immunosorbent assays may generally take over 2 hours to complete and also requires at least 2 separate incubation and washing steps. The methods and/or kits of the present disclosure may in some embodiments allow the assay to be performed in a shorter time and in some embodiments, allows a single incubation, one wash assay to be performed that is superior to previous ELISAs. For example, the reduction in handling steps may allow a reduction in common sources of variation that are introduced by multiple handling steps, plate washing, and extra pipetting steps. Therefore, such previous assays require more handling, take more time and/or use more product resources and can result in greater costs.

In addition, as a result of the multiple incubation steps and sequential addition of components, ELISA generally also requires multiple wash steps to remove unbound components after each incubation step. For example, it is not uncommon in an ELISA for washing steps to be performed after binding of a capture antibody to a solid substrate, after addition of an analyte, and after addition of a detection antibody.

In some embodiments, the methods and/or kits of the present disclosure allow the number of washing steps to be reduced compared with previous assays. For example, in some embodiments the capture agent, the analyte and the detectable agent may be added to the solid substrate at the same time, or substantially the same time, intermediate washing steps may be avoided. The reduced number of washes may allow the methods and/or kits in some embodiments to be performed in a simpler and more time-efficient manner. Furthermore, in some embodiments the reduced number of washes allows the methods and/or kits to be used for capture agents that may have a low or lower binding affinity to the analyte, as the reduced amount of washing may reduce and/or eliminate dissociation between the capture agent and the analyte.

For example, in some embodiments the reduced number of washes allows the methods and/or kits to be used for antibodies that may have a low or lower binding affinity to the analyte, as the reduced amount of washing may reduce and/or substantially eliminate dissociation between the antibody and the analyte.

Further, in previous assays that involve binding of a capture antibody to a solid substrate prior to exposure of the capture antibody to an analyte, the capture antibody is bound or adsorbed to a solid substrate in random orientations. As some of these orientations may mask part, or all, of the analyte binding domain of the capture agent, some of the capture agent bound to the solid substrate may not be available for analyte binding, thereby reducing the efficiency of the capture agent and the assay. Furthermore, in some orientations, although the capture agent may still be able to bind to the analyte, subsequent events such as binding of the detectable agent to the analyte, may be sterically hindered as a result of the orientation of the capture antibody on the solid substrate, thereby reducing the signal generated and hence the sensitivity and efficiency of the assay.

In contrast, in some embodiments the method of the present disclosure may promote the formation of a complex between a capture agent, an analyte and a detectable agent before or concurrent with contacting the complex with the solid substrate, which may prevent or inhibit binding of the capture agent to the solid substrate in an orientation which is not amenable to analyte binding. Thus, a greater proportion of the capture agent used may be available for analyte binding.

In embodiments where a peptide tag is used as a ligand and an anti peptide antibody is used as an immobilisation agent (a peptide/antibody capture system), such a system may also provide one or more additional advantages.

For example, it has been determined that a peptide/antibody capture system may have advantage over a streptavidin/biotin capture system in some embodiments, as peptide/antibody capture systems may provide one or more of increased signal, reduced variability, and reduced interference depending on the sample type.

In addition, in embodiments utilising a reduced number of washing steps of the solid substrate, a peptide/antibody capture system may provide an advantage, particularly in embodiments where the solid substrate is only washed after a complex has been immobilised. Further, in embodiments utilising a reduced time of the assay, a peptide/antibody capture system may also provide an advantage to assist in reducing assay time.

Other advantages of a peptide/antibody capture system are described herein. For example, specific peptides can be designed and prepared that are not naturally occurring, at least for the organism in which an analyte is to be detected. Bioinformatics may be used to select sequences that are unique.

Different peptides may also be selected for different applications or assays. As such, they are readily expandable if more than one affinity system is required. For example, in embodiments relating to the detection of an analyte in different wells of an assay plate, each well may be coated with a specific subset of anti-peptide antibodies which would allow the specific immobilisation of particular capture antibodies from a mixture of such antibodies with different peptide tags.

Further, in some embodiments the use of a peptide/antibody capture system may provide one or more advantages over other types of capture systems. For example, the use of a peptide/antibody system in certain embodiments may also provide an advantage over capture systems utilising poly-charged ligands (for example His tags) and metal ions (for example Ni²⁺ ions), as the peptide/antibody system may have greater affinity and/or be less likely to be affected by the presence of other charges molecules. Similarly, the use of a peptide/antibody system in certain embodiments may provide an advantage over glutathione/GST systems in that the peptide/antibody capture system also has greater affinity.

A peptide/antibody capture system may also provide in some embodiments one or more advantages over the use of anti-species antibodies as an immobilisation agent, since the system is then not restricted to the use of species of antibodies immobilised on the surface. For example, an anti-rabbit immobilised antibody can only be used to bind to rabbit capture antibodies. In addition, anti-species antibodies may suffer from reduced specificity to the species of antibody they are designed to bind, which may minimize their utility in assays using samples containing endogenous antibodies such as serum and plasma, as these will block the binding of assay antibodies.

Further, a peptide/antibody capture system may also provide in some embodiments one or more advantages over capture systems utilising immobilised protein A and/or protein G capture type systems. For example, proteins A and G will bind many antibodies in a solution. Protein A and protein G may also demonstrate reduced utility in samples containing endogenous antibodies, such as serum or plasma, as these may block binding of antibodies. In addition, such a capture system may have disadvantages in embodiments where both the capture agent and the detectable agent are antibodies, since Protein A or Protein G will not discriminate between the capture and detection antibodies, and will bind both, therefore eliminating the assay discrimination for analyte.

As described herein, certain embodiments provide methods and/or kits for detecting an analyte with a reduced assay time.

In certain embodiments, the detection of the analyte is achieved in a time of less than 120 minutes. Certain embodiments provide methods and/or kits for detecting an analyte in a sample, wherein the detection of the analyte is achieved in a time of less than 30, 40, 50, 60, 70, 80 or 90 minutes. In certain embodiments, the detection of the analyte is achieved in a time range of 30 to 90 minutes, 30 to 80 minutes, 30 to 70 minutes, 30 to 60 minutes, 40 to 80 minutes, 40 to 70 minutes, 40 to 60 minutes, 50 to 80 minutes, 50 to 70 minutes, or 50 to 60 minutes. In certain embodiments, the detection of the analyte is achieved in a time of at least 30, 40, 50, 60, 70, 80 or 90 minutes.

Certain embodiments provide methods and/or kits for detecting of an analyte in a sample, wherein the detection is achieved in a time of less than 30 minutes. In certain embodiments, the detection of the analyte is achieved in a time of less than 25, 15, 10 or 5 minutes. In certain embodiments, the detection of the analyte is achieved in a time range of 5 to 30 minutes, 5 to 25 minutes, 5 to 20 minutes, 5 to 15 minutes, 10 to 30 minutes, 10 to 25 minutes, 10 to 15 minutes, 15 to 30 minutes, 15 to 25 minutes, 15 to 20 minutes, 20 to 30 minutes, or 20 to 25 minutes. In certain embodiments, the detection of the analyte is achieved in a time of at least 5, 10, 15, 20, 25, or 30 minutes.

Certain embodiments provide methods and/or kits for detecting an analyte, wherein the detection of the analyte is achieved in a time of less than 120 minutes from contacting the sample, the capture agent, the detectable agent and the solid substrate. Certain embodiments provide methods and/or kits for detecting an analyte in a sample, wherein the detection of the analyte is achieved in a time of less than 30, 40, 50, 60, 70, 80 or 90 minutes from contacting the sample, the capture agent, the detectable agent and the solid substrate. In certain embodiments, the detection of the analyte is achieved in a time range of 30 to 90 minutes, 30 to 80 minutes, 30 to 70 minutes, 30 to 60 minutes, 40 to 80 minutes, 40 to 70 minutes, 40 to 60 minutes, 50 to 80 minutes, 50 to 70 minutes, or 50 to 60 minutes, from contacting the sample, the capture agent, the detectable agent and the solid substrate. In certain embodiments, the detection of the analyte is achieved in a time of at least 30, 40, 50, 60, 70, 80 or 90 minutes from contacting the sample, the capture agent, the detectable agent and the solid substrate.

Certain embodiments provide methods and/or kits for detecting of an analyte in a sample, wherein the detection is achieved in a time of less than 30 minutes from contacting the sample, the capture agent, the detectable agent and the solid substrate. In certain embodiments, the detection of the analyte is achieved in a time of less than 25, 15, 10 or 5 minutes from contacting the sample, the capture agent, the detectable agent and the solid substrate. In certain embodiments, the detection of the analyte is achieved in a time range of 5 to 30 minutes, 5 to 25 minutes, 5 to 20 minutes, 5 to 15 minutes, 10 to 30 minutes, 10 to 25 minutes, 10 to 15 minutes, 15 to 30 minutes, 15 to 25 minutes, 15 to 20 minutes, 20 to 30 minutes, or 20 to 25 minutes from contacting the sample, the capture agent, the detectable agent and the solid substrate. In certain embodiments, the detection of the analyte is achieved in a time of at least 5, 10, 15, 20, 25, or 30 minutes from contacting the sample, the capture agent, the detectable agent and the solid substrate.

As described herein, certain embodiments provide methods and/or kits for detecting an analyte using a capture agent and/or a detectable agent with reduced affinity for the analyte.

In certain embodiments, the methods and/or kits of the present disclosure may be used with a capture agent and/or a detectable agent having a Kd for binding with the analyte of greater than 10-M. In further embodiments, the capture agent and/or detectable agent has a Kd for binding with the analyte of greater than 10⁻⁷M, 10⁻⁸M or 10⁻⁹M. In certain embodiments, the Kd is in the range from 10⁻⁸M to 10⁻¹²M. In certain embodiments, the capture agent and/or detectable agent are an antibody.

As described herein, certain embodiments provide methods and/or kits for detecting an analyte providing improved specificity and/or sensitivity. In certain embodiments, the sensitivity is improved by the formation of a complex between a capture agent, an analyte and a detectable agent before or concurrent with contacting the complex with the solid substrate

As described herein, the methods and/or kits of the present disclosure allow for the detection of an analyte in a sample. In this regard, it will be understood that some embodiments of the present disclosure represent an immunoassay. Some embodiments of the present disclosure represent a sandwich assay, in which the analyte is bound to a capture antibody and to an antibody detectable agent.

In certain embodiments, the methods and/or kits for detecting an analyte in a sample may comprise a qualitative determination of whether the analyte is present or absent in the sample.

In certain embodiments, the methods and/or kits may comprise a quantitative assessment of the levels of the analyte in the sample. For example, in certain embodiments the methods and/or kits allow for the quantification of the concentration of the analyte in the sample. Methods for the calculation of the concentration of an analyte are known.

In certain embodiments, the method for detecting an analyte comprises an immunoassay. In certain embodiments, the immunoassay comprises a non-competitive immunoassay. In certain embodiments, the immunoassay comprises a competitive immunoassay. In certain embodiments, the immunoassay comprises a combination of both a non-competitive and a competitive immunoassay.

As described herein, in certain embodiments one or more analytes are detected. In certain embodiments, one or more analytes are detected in the same reaction vessel.

In certain embodiments, the analyte is an analyte comprising one or more antigenic sites that allow the analyte to be bound by an antibody capture agent and/or an antibody detectable agent.

In certain embodiments, the analyte is a non-nucleic acid analyte.

Certain embodiments are based on the capture of the analyte by the capture agent via a mechanism that is not substantially based on nucleic acid-nucleic acid interactions, such as binding based on complementary base pairing. That being said, it will be understood that in some embodiments, the analyte may comprise a nucleic acid component. In certain embodiments the binding of the analyte by the capture agent is substantially based on hydrophobic, hydrophilic, polyanionic-polycationic, van der Waals, or combinations thereof, substantially exclusive of nucleic acid-nucleic acid interactions.

Examples of analytes that may be detected by the methods of the present disclosure comprise a microbe, a virus, a protein, a macromolecule, a small molecule, a drug or combinations thereof. Other types of analyte are contemplated. In certain embodiments, the analyte comprises a protein. In certain embodiments, the analyte comprises a component of a cell signalling pathway, a cytokine, a tumour suppressor, an antibody or a fragment thereof, or combinations thereof.

In certain embodiments, the analyte may comprise a particular form or state of a molecule, such as a protein. For example, in certain embodiments, the method may be used to detect a protein that is phosphorylated, methylated, glycosylated or combinations thereof. In these embodiments, at least one of the capture agent and the detectable agent may have specificity to only one form of the protein (for example the capture agent may only bind to the phosphorylated form of the protein and not to the unphosphorylated form of the protein).

In certain embodiments, the analyte may comprise a phosphoprotein. Examples of phosphoproteins comprise phosphorylated ERK, S6 p240/44, AKT pT308 or AKT pS473. In certain embodiments, the analyte is selected from the group consisting of phospho-ERK 1/2; total ERK 1/2; phospho-Akt 1/2/3; total Akt 1/2/3; phospho-NF-Kβ p65; total NF-Kβ 65; phospho-1-kBα; total-kBα; phospho-STAT3; total STAT3; phospho-STATS A/B; phospho-JNK 1/2/3; total JNK 1/2/3; phospho-p38 MAPKα; total p38 MAPKα; phospho-p53; total p53; phospho-p70S6K; total p70S6K; and GAPDH.

In certain embodiments, the analyte is present in the sample at a concentration of 100 ng/ml or less, 10 ng/ml or less, 1 ng/ml or less, 100 pg/ml or less, or 10 pg/ml or less, 1 pg·ml or less, 100 fg/ml or less, 10 fg/ml or less, or 1 fg/ml or less. In certain embodiments, the analyte is present in the sample at a concentration of greater than 100 ng/ml, greater than 10 ng/ml, greater than 1 ng/ml, greater than 100 pg/ml or, greater than 10 pg/ml, greater than 1 pg/ml, greater than 100 fg/ml, greater than 10 fg/ml or greater than 1 fg/ml. In certain embodiments the analyte is present in the sample at a concentration of between 1 fg/ml to 100 ng/ml, 1 fg/ml to 10 ng/ml, 1 fg/ml to 1 ng/ml, 10 fg/ml to 100 ng/ml, 10 fg/ml to 10 ng/ml, 10 fg/ml to 1 ng/ml, 100 fg/ml to 100 ng/ml, 100 fg/ml to 10 ng/ml, 100 fg/ml to 1 ng/ml, 1 pg/ml to 100 ng/ml, 1 pg/ml to 10 ng/ml, or 1 pg/ml to 1 ng/ml. Other concentrations are contemplated.

As described herein, the present disclosure provides methods and/or kits for detecting an analyte in a sample.

In certain embodiments, the sample comprises a mixture, composition, solution, that may or may not contain an analyte. In certain embodiments, the sample comprises one or more samples. In some embodiments, the sample comprises one or more samples comprising one or more analytes to be detected. In certain embodiments, the sample comprises a laboratory or research sample, a medical sample, a biological sample, a cell sample, a water sample, a food sample, and an agricultural sample, a spiked sample, a derivative of these samples or combinations thereof. In certain embodiments, the sample comprises a medical sample or a cell sample, such as a blood sample, a serum sample, a urine sample, a milk sample, a cell lysate, a derivative of these samples and/or combinations thereof.

In certain embodiments, the sample may be pre-treated before being used in the methods and/or kits. For example, the sample may be pre-cleared, concentrated, diluted, induced, pre-treated or processed to remove one or more components or impurities from the sample using methods known.

In relation to biological samples, a particular deficiency of many previous methods for detecting proteins is that they are not suitable for use with samples with moderate to high levels of proteins (for example as often found in samples comprising serum or cell lysates). In certain embodiments, the methods and/or kits disclosed are able to handle such samples. For example, in certain embodiments the utilisation of the immobilisation agent allows the method to be performed in the presence of moderate or high protein concentrations. Moderate or high protein concentrations may also be introduced, for example, during blocking steps or may be included in the sample itself.

For example, the sample may comprise a serum sample which may have a protein concentration up to approximately 60-80 mg/ml, a cell lysate sample which may have a protein concentration of approximately 1-3 mg/ml, or a sample from a cell-based assay which may include protein contamination from fetal bovine serum (FBS), or the like, which may be used in cell culture media. Protein contamination from media may also account for 1-5% of the final protein contamination in a cell lysate, which may translate to approximately 0.6-4 mg/ml of protein in addition to the cellular protein.

In certain embodiments, the sample may comprise a protein concentration of more than 0.01 mg/ml, a protein concentration of more than 0.1 mg/ml, a protein concentration of more than 1 mg/ml, a protein concentration of more than 2 mg/ml, a protein concentration of more than 10 mg/ml, or a protein concentration of more than 60 mg/ml. In certain embodiments, the sample may comprise a protein concentration of less than 0.01 mg/ml, a protein concentration of less than 0.1 mg/ml, a protein concentration of less than 1 mg/ml, a protein concentration of less than 2 mg/ml, a protein concentration of less than 10 mg/ml, or a protein concentration of less than 60 mg/ml.

In certain embodiments, the methods and/or kits of the present disclosure are compatible for analyte detection in a range of biological milieu, such as cellular lysates, and/or serum.

As described herein, the methods and/or kits for detecting an analyte in a sample comprise providing a reaction vessel, being for example a physical container that allows the contacting of the sample, the capture agent, the detectable agent and the solid substrate to occur in the container. Examples of reaction vessels include a test tube, a micro centrifuge tube, a well, or a flask. In some embodiments, the reaction vessel comprises a well of a multi-well plate, such as a microtitre plate, or a well or surface of a microfluidic device. Multi-well plates are an example of an assay platform comprising a plurality of reaction vessels.

In certain embodiments, one or more reaction vessels are utilised to detect the analyte. In certain embodiments, a single reaction vessel is utilised to detect the analyte. In certain embodiments, a single reaction vessel is utilised for performing the steps in the method onward from the contacting of the sample, the antibody capture agent, the detectable agent and the solid substrate. In certain embodiments this may reduce the handling steps involved in the method as compared to previous assays and thereby provide an improvement over such assays, including the ability to provide more consistent results over such assays. In certain embodiments, a single reaction vessel is utilised to detect one or more analytes. In certain embodiments, a single reaction vessel is utilised to detect a plurality of analytes.

In certain embodiments, the one or more reaction vessels comprise the same solid substrate. In certain embodiments, the one or more reaction vessels comprise one or more solid substrates.

In certain embodiments, the one or more reaction vessels comprise the same immobilisation agent. In certain embodiments, the one or more reaction vessels comprise at least two different immobilisation agents. In certain embodiments, the one or more reaction vessels comprise one or more bound immobilisation agents. In certain embodiments, an assay platform comprises one or more reaction vessels comprising one or more solid substrates. In certain embodiments, the assay platform comprises one or more reaction vessels comprising one or more immobilisation agents.

In certain embodiments, the assay platform comprises a multi-well plate, such as a microtitre plate. In certain embodiments, the assay platform comprises a plurality of reaction vessels comprising a solid substrate comprising the bound immobilisation agent. In certain embodiments, the assay platform comprises a plurality of reaction vessels comprising a solid substrate comprising the same bound immobilisation agent. In certain embodiments, the assay platform comprises a plurality of reaction vessels, one or more of the plurality of reaction vessels comprising the same bound immobilisation agent.

In certain embodiments, the methods and/or kits of the present disclosure provide one or more capture agents, the one or more capture agents being able to bind to one or more analytes to be detected.

As described herein, in certain embodiments, the use of a biotin-steptavidin/avidin capture system in conjunction with no additional washing of the solid substrate after contacting of the solid substrate with any one or more of the sample, the capture agent and the detectable agent may provide an advantage to the detection of more than one analytes.

As described herein, the methods and/or kits of the present disclosure provide a solid substrate comprising a bound immobilisation agent.

In certain embodiments, the reaction vessel comprises the solid substrate. In certain embodiments, the solid substrate may be part of the reaction vessel. For example, the solid substrate may be integral with substantially all or part of the reaction vessel, and/or the solid substrate may form part of the surface of the reaction vessel (such as the surface of a well of a microtitre plate) or may be attached to the reaction vessel. Other combinations are also contemplated.

In certain embodiments, the solid substrate is separate to the reaction vessel. In these embodiments, the solid substrate may be mobilisable and may be added to the reaction vessel. For example, the solid substrate may be a bead, an affinity matrix, a resin, a gel, a slurry, a strip, or a dip stick. Combinations of different types of substrates are also contemplated.

Beads and their use are known. In certain embodiments, the bead is a magnetic bead. Magnetic beads are known and commercially available. Methods for the use of magnetic beads are known.

In certain embodiments, the immobilisation agent is bound to the solid substrate by a covalent attachment to the solid substrate. For example, in certain embodiments wherein the solid substrate is a bead, the immobilisation agent may be bound to the bead by a covalent attachment to the bead. In certain embodiments, the immobilisation agent is actively bound to the solid substrate.

In certain embodiments, the immobilisation agent is bound to the solid substrate via a non-covalent attachment. Examples of such interactions include a hydrophilic interaction, a hydrophobic interaction, a charged (ionic) interaction, a van de Waals interaction, or combinations of such interactions. In certain embodiments, the immobilisation agent is passively bound to the solid substrate, such as an antibody being coated onto a solid substrate.

In certain embodiments, the solid substrate is all or part of a well. In certain embodiments, the solid substrate is all or part of a well of a multi-well plate, such as a microtitre plate. In certain embodiments, the solid substrate is all or part of a tip, such as a pipette tip. In certain embodiments, the solid substrate is all or part of a bead, such as bead used for FACS analysis and/or a magnetic bead.

The use of a bound immobilisation agent on the solid substrate in the methods and/or kits of the present disclosure provides flexibility in the selection of the substrate that may be used. For example, the immobilisation agent may allow a particular capture agent to bind to a solid substrate (via the immobilisation agent) to which it would otherwise not bind. Moreover, the use of an immobilisation agent-ligand binding pair allows the method to be modular in that a range of capture agents may be produced that bind to a particular solid substrate by incorporation of a ligand for the immobilisation agent on the solid substrate into the capture agents. As described herein, in certain embodiments the use of a bound immobilisation agent allows the use of different immobilisation agents for binding different capture agents.

In certain embodiments, the solid substrate may comprise a substance that promotes binding of the immobilisation agent or may be treated to promote binding of the immobilisation agent. In certain embodiments, the solid substrate may comprise a plastic surface including, for example, a polystyrene surface, a polyvinyl chloride surface or a cyclo-olefin surface. In certain embodiments, the solid substrate may be transparent or coloured depending whether the detection method involves a colorimetric, fluorescence or other forms of read outs. In certain embodiments, the solid substrate may comprise a hydrophobic surface.

In certain embodiments, the solid substrate may be treated to increase the binding affinity of the immobilisation agent to the solid substrate. For example, the solid substrate may be irradiated and/or functionalised to allow covalent bonding between the substrate and the immobilisation agent.

The use of a bound immobilisation agent provides one or more advantages to certain embodiments of the methods and/or kits for detecting an analyte. For example, the use of a bound immobilisation agent diminishes the potential influence of substrate-reactive proteins in the sample. For example, hydrophobic proteins in the sample are less likely to affect the outcome of the method even if it is performed on a hydrophobic solid substrate, as the immobilisation agent is already bound to the solid substrate and the ligand on the capture agent may be specific for the immobilisation agent.

In certain embodiments, the immobilisation agent comprises an antibody, avidin and/or steptavidin and/or derivatives thereof, or one member of a binding pair as described herein.

In certain embodiments, the immobilisation agent comprises an antibody. In certain embodiments, a solid substrate may be coated with an antibody using a concentration of antibody of 0.1 ug/ml or greater, 0.25 ug/ml or greater, 0.5 ug/ml or greater, 1.0 ug/ml or greater, 2.5 ug/ml or greater, 5 ug/ml or greater, 10 ug/ml or greater or 25 ug/ml or greater. In certain embodiments, the solid substrate may be coated with an antibody at a concentration of 0.1 ug/ml or less, 0.25 ug/ml or less, 0.5 ug/ml or less, 1.0 ug/ml or less, 2.5 ug/ml or less, 5 ug/ml or less, 10 ug/ml or less or 25 ug/ml or less. In certain embodiments, the solid substrate may be coated with an antibody in the concentration range from 0.1-25 ug/ml, 0.1-10 ug/ml, 0.1-5 ug/ml, 0.1-1 ug/ml, 0.1-0.5 ug/ml, 0.25-25 ug/ml, 0.25-10 ug/ml, 0.25-5 ug/ml, 0.25-1 ug/ml, 0.25-0.5 ug/ml, 0.5-25 ug/ml, 0.5-10 ug/ml, 0.5-5 ug/ml, 0.5-2.5 ug/ml, 0.5-1.0 ug/ml, 1-25 ug/ml, 1-10 ug/ml, 1-5 ug/ml, 1-2.5 ug/ml, 2.5-25 ug/ml, 2.5-10 ug/ml, 2.5-5 ug/ml, 5-25 ug/ml, 5-10 ug/ml, or 10-25 ug/ml.

In certain embodiments, the immobilisation agent and the ligand on the capture agent form a binding pair. A range of different immobilisation agent and ligand binding pairs may be used.

In certain embodiments, the immobilisation agent and ligand may be interchangeable (i.e. a first compound may be bound to the solid substrate or the capture agent and a second compound, which is part of the same binding pair, may be bound to the other).

In certain embodiments, the immobilisation agent and the ligand are a binding pair that is not a polyanionic-polycationic binding pair. In certain embodiments the use of an immobilisation agent-ligand binding pair which do not bind substantially through an ionic interaction between a substantially polyanionic molecule and a substantially polycationic molecule may provide one or more advantages to the method and/or kits for detecting an analyte in a sample. Examples of such polyionic molecules include polymeric ionic substances, or polypeptides with repeated charged amino acids, such as a polyhistidine tag.

For example, advantages of using a immobilisation agent-ligand binding pair that is not a polyanionic-polycationic binding pair include, for example, the fact that the binding between the pair of molecules is less dependent upon the pH of any solution contacting the binding pair and/or the fact that the ability to reduce non-specific interactions is difficult with such polyionic binding pairs. Furthermore, many proteins present in biological milieu may specifically bind either polyanions or polycations, making these components potentially difficult to detect with such an immobilisation system.

In addition, in certain embodiments the use of a immobilisation agent-ligand binding pair that is not a polyanionic-polycationic binding pair may provide other advantages including promoting the formation of a complex between the capture agent, the analyte and the detectable agent, may improve the access of such a complex to the solid substrate and may promote the ability of the detectable agent to access the analyte for detection purposes.

In certain embodiments, the immobilization agent-ligand binding pair comprises a peptide tag used as a ligand and an immobilization agent that may bind to the peptide tag. In certain embodiments, the immobilization agent-ligand binding pair comprises a peptide tag and an anti-peptide tag antibody.

As described herein, peptide-tags are polypeptide protein tags that can be conjugated to another molecule, such as a protein (eg an antibody) or added to a protein using recombinant DNA technology. One example of a peptide tag is the octapeptide DYKDDDDK (SEQ ID NO. 1), otherwise referred to as a FLAG-tag, which can be used in different assays that utilize recognition by an antibody. Other examples of peptide tags are described herein. In certain embodiments, the peptide tag does not comprise a plurality of consecutive amino acids with the same charge.

A range of anti peptide tag antibodies may be obtained or produced by a person skilled in the art. For example, commercially available anti-DYKDDDDK (SEQ ID NO.1) antibodies are described herein. Commercially available anti-DYKDDDDK (SEQ ID NO.1) antibodies include Sigma-Aldrich product codes F7425, F3040, F1804, F3165, F4042, F2555 and SAB4200071. Some commercially available antibodies recognize the DYKDDDDK (SEQ ID NO.1) tag only in certain positions on a protein, for example exclusively N-terminal. However, other available antibodies are position-insensitive.

In certain embodiments, the peptide tag comprises a peptide derived from a member of a signalling pathway and/or a peptide from a cytokine. Peptide tags are as described herein, and the addition of a peptide tag to the capture agent to form a conjugate may be achieved by a suitable known method. Other peptide tags are contemplated. Peptide tags may be naturally occurring or non-naturally occurring. In certain embodiments, the peptide tag has greater than 75%, 80%, 85%, 90% or 95% sequence identity to a naturally occurring polypeptide sequence. In certain embodiments, the peptide tag has greater than 75%, 80%, 85%, 90% or 95% sequence homology to a naturally occurring polypeptide sequence. Methods for determining sequence identity and sequence homology are known.

In certain embodiments, the peptide tag comprise KRITVEEALAHPYLEQYYDPTDE (SEQ ID NO.2), being a sequence derived from the carboxy terminus of the human ERK proteins (ERK C-term peptide). Antibodies to this peptide tag may be produced by known methods.

Adding a peptide tag to a protein allows the protein to be bound and/or immobilised by an agent that binds the peptide tag, for example an antibody raised against the peptide tag sequence. In certain embodiments, a peptide tag may also be used in conjunction with other affinity tags for example a polyhistidine tag (His-tag), HA-tag or myc-tag.

In certain embodiments, the use of a peptide tag conjugated to a capture agent and an anti-peptide antibody (as the immobilization agent) may provide one or more advantages over other types of immobilization agent-ligand binding pairs. For example, in certain embodiments, peptide tag and anti-peptide antibodies may be less susceptible to variation over different sample types and/or provide improved sensitivity of detection.

In certain embodiments, the immobilisation agent and ligand binding pairs comprise biotin and avidin or streptavidin (or derivates thereof); metal chelate (e.g. copper, nickel, cobalt) and Histidine (e.g. histidine tagged proteins); maleic anhydride and amine (e.g. amine containing proteins); or meleimide and sulfhydryls (e.g. sulfhydryl peptides).

As discussed herein, in certain embodiments the immobilisation agent comprises avidin, streptavidin and/or derivatives thereof and the ligand comprises biotin or derivates thereof. In certain embodiment, the immobilisation agent and the ligand of this binding pair are interchanged. Derivatives of avidin or streptavidin are known and may include forms of avidin or streptavidin that have been modified to increase their binding affinity to modified and/or unmodified solid substrates or ligands. For example, streptavidin may be modified to add one or more amine groups, histidine residues or sulfhydryl groups to the molecule. In some embodiments, the derivative of streptavidin may comprise neutravidin, captavidin or streptavidin mutants (e.g. H127C or S139C).

As discussed herein, in some embodiments where a peptide tag is used as a ligand, the corresponding immobilization agent may comprise an anti-peptide antibody. In certain embodiment, the immobilisation agent and the ligand of this type of binding pair are interchanged. A range of anti-peptide antibodies may be obtained or produced by a skilled person.

In certain embodiments, hydrophobic or hydrophilic immobilisation agents may be passively bound to the hydrophobic or hydrophilic solid substrates, respectively. For example, antibodies may be passively bound to a hydrophobic solid substrate, or streptavidin (or derivates thereof) may be passively bound to a hydrophobic solid substrate.

In certain embodiments, the solid substrate may comprise a linker which facilitates covalent bonding of the immobilisation agent to the solid substrate. For example, the linker may comprise glutathione, maleic anhydride, a metal chelate, or maleimide. The immobilisation agent may then be bound to the solid substrate via the linker.

In certain embodiments, the solid substrate comprising the bound immobilisation agent may be treated with a blocking agent that binds non-specifically to and substantially saturates binding sites to prevent unwanted binding of ligand or other components to the excess sites on the solid substrate. In certain embodiments, a blocking agent may be included during the binding reactions. Examples of blocking agents may include gelatin, BSA, egg albumin, casein, and non-fat milk. In certain embodiments, the solid substrate and/or the bound immobilisation agent may be treated with the blocking agent prior to the addition of the capture agent and/or concurrent with the addition of the capture agent.

In certain embodiments the methods and/or kits of the present disclosure comprise providing an antibody capture agent which can bind the analyte, wherein the capture agent comprises a ligand for the immobilisation agent. In certain embodiments, the methods and/or kits of the present disclosure comprises providing an antibody detectable agent which can bind the analyte.

The term “antibody” is to be understood to mean an immunoglobulin molecule with the ability to bind an antigenic region of another molecule, and includes monoclonal antibodies, polyclonal antibodies, multivalent antibodies, chimeric antibodies, multispecific antibodies, diabodies and fragments of an immunoglobulin molecule or combinations thereof that have the ability to bind to the antigenic region of another molecule with the desired affinity including a Fab, Fab′, F(ab′)2, Fv, a single-chain antibody (scFv) or a polypeptide that contains at least a portion of an immunoglobulin that is sufficient to confer specific antigen binding, such as a molecule including one or more CDRs. Antibodies to specific analytes may be obtained commercially or generated by known methods. For example, antibodies to specific analytes may be prepared using known methods.

As described herein, in certain embodiments the methods and/or kits of the present disclosure comprise providing the capture agent in solution.

In many previous methods for detecting an analyte using a capture agent, the capture agent is immobilised on the solid substrate prior to coming into contact with the analyte and binding to the analyte. In certain embodiments, the use of a capture agent in solution provides one or more advantages over the use of a capture agent immobilised on the solid substrate. Without being bound by theory, it is believed that the use of the capture agent in solution in certain disclosed embodiments disclosure promotes the binding of the capture agent to the analyte and thereby promotes the formation of a complex of the capture agent, the analyte and the detectable agent.

In certain embodiments the present disclosure provides a method for detecting an analyte in a sample, the method comprising:
providing a reaction vessel;
providing a solid substrate comprising a bound immobilisation agent;
providing a capture agent in solution which can bind the analyte, wherein the capture agent comprises a ligand for the immobilisation agent;
providing a detectable agent which can bind to the analyte;
contacting the sample, the capture agent, the detectable agent and the solid substrate in the reaction vessel;
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

In this regard, it will be understood that in some embodiments the capture agent is provided in solution prior to contacting the capture agent with the sample. Accordingly, in certain embodiments the capture agent is provided in a substantially liquid state.

In certain embodiments, the capture agent is in solution. Examples of capture agents are as described herein. In certain embodiments, the capture agent comprises an antibody or an antigen binding part thereof in solution. In certain embodiments, the use of antibody capture agent in solution also allows the amount of a specific antibody to a target to be reduced as compared to previous assays, as more target-specific capture antibody is required in assays when the adsorbed capture agent is pre-adsorbed onto a plate. In certain embodiments there is no pre-immobilisation of the capture agent on the solid substrate.

As described herein, the capture agent comprises a ligand for the immobilisation agent. In this regard, immobilisation agent-ligand binding pairs are as described herein. In certain embodiments, the capture agent comprises a plurality of ligands for the immobilisation agent. In certain embodiments, the capture agent comprises different ligands. In certain embodiments, the capture agent comprises, at a plurality of sites, a ligand for the immobilisation agent. Immobilisation agent-ligand binding pairs are as described herein.

In certain embodiments, the ligand is part of the capture agent. For example, the capture agent may comprise histidine residues, amine groups or sulfhydryl groups that are able to bind to the immobilisation agent.

In certain embodiments, the ligand for the immobilisation agent comprises a peptide tag, such as a DYKDDDDK (SEQ ID NO.1) tag. As described herein, peptide tags are polypeptide protein tags that can be conjugated to another molecule, such as a protein. In certain embodiments, a peptide tag may be conjugated to an antibody or added to a protein using recombinant DNA technology. One example of a peptide conjugate tag is a DYKDDDDK (SEQ ID NO.1) tag, which can be used in different assays that utilize recognition by an antibody. Other examples of peptide tags comprise a polypeptide sequence comprising one or more of the following sequences: HHHHHH (SEQ ID NO.3); EQKLISEEDL (SEQ ID NO.4); YPYDVPDYA (SEQ ID NO.5); YTDIEMNRLGK (SEQ ID NO.6); and QPELAPEDPED (SEQ ID NO.7).

In certain embodiments, adding a peptide tag to a protein allows the protein to be bound and/or immobilised by an antibody against the peptide conjugate sequence or immobilized by another type of agent that can bind to the peptide. In some embodiments, a peptide tag may also be used in conjunction with other affinity tags.

As described herein, the addition of a peptide tag to the capture agent may be achieved by a suitable known method. In certain embodiments, the ligand is bound to the capture agent. For example, in certain embodiments, the ligand may be amine reactive, carbohydrate reactive, carboxyl reactive, or sulfhydryl reactive and thus may bind to the capture agent via primary amines (e.g. lysine or the N-terminus), carbohydrate modifications, carboxyl groups (e.g. on aspartic acid residues, glutamic acid residues and the C-terminus), or sulfhydryl groups. In certain embodiments, the ligand may comprise iodinatable and/or photoactivatable groups. In certain embodiments, the ligand may comprise tetrafluorophenyl azide (TFPA) groups that, once activated by UV light, are able to covalently bind at sites containing C—H or N—H bonds (e.g. the ligand may comprise TFPA-PEG3-Biotin). Methods for labelling proteins and other molecules with the above ligands are known.

In certain embodiments, the ligand comprises biotin or a derivative thereof, for example iminobiotin, D-desthiobiotin, DSB-X-biotin, biotin dimers or arylstannyl-biotin trimer. Biotin and derivatives thereof may be bound to the capture agent by biotinylation. Biotinylation reagents and methods for biotinylation of a target molecule are known in the art. Biotinylation may comprise, for example, primary amine biotinylation, sulfhydryl biotinylation, carboxyl biotinylation, or glycoprotein biotinylation.

In certain embodiments, the capture agent comprises a plurality of ligands for the immobilisation agent. In certain embodiments, the use of a capture agent comprising a plurality of ligands for the immobilisation agent may assist in the formation and/or detection of a complex between the capture agent, the analyte and the detectable agent. In addition, in certain embodiments the use of a capture agent comprising a plurality of ligands may reduce the amount of capture agent that binds to the immobilisation agent in an orientation that masks the analyte binding domain.

In certain embodiments as a result of more efficient use of the capture agent, the amount of capture agent that is required to produce a given amount of detectable signal may also be reduced relative to previous methods, such as previous ELISAs. In addition, more efficient use of the capture agent may also lead to a reduction in the area of solid substrate required to produce a given level detectable signal relative to previous assays, such as conventional ELISA.

In certain embodiments, the methods and/or kits comprise a reduced amount of a capture agent, relative to previous assays, as a result of the more efficient binding of a capture to the solid substrate. For example, the capture agent may be present at a concentration of 1000 ng/ml or less, 900 ng/ml or less, 800 ng/ml or less, 700 ng/ml or less, 600 ng/ml or less, 500 ng/ml or less, 400 ng/ml or less, 300 ng/ml or less, 200 ng/ml or less, 100 ng/ml or less, 50 ng/ml or less, 25 ng/ml or less, or 10 ng/ml or less. In some embodiments, the antibody capture agent may be present at a concentration of 10 ng/ml or greater, 25 ng/ml or greater, 50 ng/ml or greater, 100 ng/ml or greater, 200 ng/ml or greater, 300 ng/ml or greater, 400 ng/ml or greater, 500 ng/ml or greater, or 600 ng/ml greater, 700 ng/ml or greater, 800 ng/ml or greater, 900 ng/ml or greater, 1000 ng/ml or greater. In some embodiments, the antibody capture agent is present at a concentration of 10 ng/ml to 1000 ng/ml, 10 ng/ml to 900 ng/ml, 10 ng/ml to 800 ng/ml, 10 ng/ml to 700 ng/ml 10 ng/ml to 600 ng/ml, 10 ng/ml to 500 ng/ml, 10 ng/to 400 ng/ml, 10 ng/ml to 200 ng/ml, 10 ng/ml to 100 ng/ml, 25 ng/ml to 1000 ng/ml, 25 ng/ml to 900 ng/ml 25 ng/ml to 800 ng/ml, 25 ng/ml to 700 ng/ml, 25 ng/ml to 600 ng/ml, 25 ng/ml to 500 ng/ml, 25 ng to 400 ng/ml, 25 ng/ml to 200 ng/ml, 25 ng/ml to 100 ng/ml, 50 ng/ml to 1000 ng/ml, 50 ng/ml to 900 ng/ml, 50 ng/ml to 800 ng/ml, 50 ng/ml to 700 ng/ml, 50 ng/ml to 600 ng/ml, 50 ng/ml to 500 ng/ml, 50 ng/to 400 ng/ml, 50 ng/ml to 200 ng/ml, or 50 ng/ml to 100 ng/ml.

In certain embodiments, the binding capacity of the at least one solid substrate for a capture agent is 200 ng/ml or greater, 500 ng/ml, 1 ug/ml or greater, 2 ug/ml or greater, 3 ug/ml or greater, 4 ug/ml or greater, or 5 ug/ml or greater. In certain embodiments, the binding capacity is 5 ug/ml or less, 4 ug/ml or less, 3 ug/ml or less, 2 ug/ml or less, 1 ug/ml or less, 500 ng/ml or less or 200 ng/ml or less. For example, in certain embodiments where a capture agent is an antibody capture agent, the binding capacity of the at least one solid substrate is typically 2 ug/ml for a standard microtitre plate. In certain embodiments, the at least one solid substrate has a binding capacity of the aforementioned amounts for protein. In certain embodiments, the binding capacity of the least one solid substrate for a capture agent is 200 ng/ml to 500 ng/ml, 200 ng/ml to 1 ug/ml, 200 ng/ml to 2 ug/ml, 200 ng/ml to 3 ug/ml, 200 ng/ml to 4 ug/ml, 200 ng/ml to 5 ug/ml, 500 ng/ml to 1 ug/ml, 500 ng/ml to 2 ug/ml, 500 ng/ml to 3 ug/ml, 500 ng/ml to 4 ug/ml, 500 ng/ml to 5 ug/ml, 1 ug/ml to 2 ug/ml, 1 ug/ml to 3 ug/ml, 1 ug/ml to 4 ug/ml, 1 ug/ml to 5 ug/ml, 2 ug/ml to 3 ug/ml, 2 ug/ml to 5 ug/ml, 3 ug/ml to 4 ug/ml, 3 ug/ml to 5 ug/ml, or 4 ug/ml to 5 ug/ml. In certain embodiments, the at least one solid substrate has a binding capacity of the aforementioned amounts for protein.

For example, in an anti-peptide conjugate system, an antibody capture agent may be typically used at a concentration of 50 ng/ml, and in a streptavidin-biotin system an antibody capture agent may typically be used at a concentration of 200 ng/ml.

As described herein, the methods and/or kits of the present disclosure comprise providing a detectable agent which can bind to the analyte.

In certain embodiments the detectable agent is provided in solution. Accordingly, in certain embodiments the detectable agent is provided in a substantially liquid state.

In certain embodiments, the present disclosure provides a method for detecting an analyte in a sample, the method comprising:
providing a reaction vessel;
providing a solid substrate comprising a bound immobilisation agent;
providing a capture agent which can bind the analyte, wherein the capture agent comprises a ligand for the immobilisation agent;
providing a detectable agent in solution which can bind to the analyte; contacting the sample, the capture agent, the detectable agent and the solid substrate in the reaction vessel;
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

As described herein, in certain embodiments, a non-nucleic acid analyte may be detected using an antibody capture agent in solution.

In certain embodiments, the present disclosure provides a method for detecting a non-nucleic acid analyte in a sample, the method comprising:
providing a reaction vessel;
providing a solid substrate comprising a bound immobilisation agent;
providing an antibody capture agent in solution which can bind the analyte, wherein the capture agent comprises a ligand for the immobilisation agent;
providing a detectable agent in solution which can bind to the analyte;
contacting the sample, the capture agent, the detectable agent and the solid substrate in the reaction vessel;
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

In certain embodiments, the use of a detectable agent in solution may provide one or more advantages to the methods and/or kits of the present disclosure, including promoting the formation and detection of a complex of the capture agent, the analyte and the detectable agent.

In certain embodiments, the methods and/or kits of the present disclosure provide one or more detectable agents, the one or more detectable agents being able to bind to one or more analytes to be detected.

In certain embodiments, the detectable agent comprises an antibody or a fragment thereof, an aptamer, or a protein receptor or ligand (or binding fragment thereof). Examples of antibodies and binding fragments thereof, aptamers, and protein receptors or ligands, are as described herein.

In embodiments where protein receptors or ligands are used as a detectable agent, these may comprise the whole receptor or ligand or a fragment thereof (for example a fragment comprising a binding domain of the receptor or ligand). In certain embodiments, the receptor or ligand (or fragment thereof) may comprise a fusion protein. Fusion partners may include, for example, fluorescent fusion partners (e.g. GFP) and immunoglobulin fusion partners. Fusion partners and methods for preparing fusion proteins are known in the art. In certain embodiments, the fusion partner may act to stabilise the receptor or ligand (or fragment thereof), provide a detectable signal (e.g. for fluorescent fusion partners) or provide a target for antibody, or other, binding or immobilisation.

Aptamers used as a detectable agent may be obtained commercially or generated by known methods.

In certain embodiments, the detectable agent may comprise a detectable tag. In certain embodiments, the detectable tag may be applied to the detectable agent (for example bound to the detectable agent) or may be part of the detectable agent (for example the detectable agent may include the detectable tag as a fusion partner, a labelled amino acid or labelled nucleotide). Examples of suitable detectable tags include antigens, enzymes, fluorophores, quenchers, radioactive isotopes, one or more lanthanide ions such as one or more of Eu³⁺, Sm³⁺, Tb³⁺, and Dy³⁺, and luminescent labels. It will be appreciated that the detectable tag may be detected directly or indirectly via a further molecule that can produce a detectable signal.

Antigens that may be used as a detectable tag may include, for example, an antigenic component of the detectable agent that may be targeted by a secondary detectable agent. For example, in some embodiments, a secondary antibody may be used to detect an antigen on a detectable agent. The secondary antibody may, for example, be fluorescently or enzymatically labelled. In embodiments where the detectable agent is a primary antibody (ie. an analyte binding antibody), the secondary antibody may have binding affinity to an antigen on the primary antibody. For example, the antigen may be derived from the host in which the detectable agent was raised.

In certain embodiments, the detectable tag comprises an enzyme. Enzymes that may be used as detectable tags include, for example, enzymes that result in the conversion of a substrate into a detectable product (generally resulting in a change in colour or fluorescence or generation of an electrochemical signal). Such enzymes may include, for example, horseradish peroxidase (HRP), alkaline phosphatase (AP), β-galactosidase, acetylcholinesterase, luciferase, catalase or combinations thereof. Depending on the enzyme and substrate used, detection may be performed with a spectrophotometer, fluorometer, luminometer, electrochemical detection means. Other means for detection are contemplated.

Radioactive isotopes that may be used as detectable tags include, for example, ³H, ¹⁴C, ³²P, ³⁵S, or ¹³¹I. Other isotopes are contemplated. The radioisotope may be conjugated to a detectable agent or incorporated into a detectable agent by translation of mRNA encoding the detectable agent in the presence of radiolabelled amino acids. Radioisotopes and methods for conjugating radioactive isotopes to molecules such as proteins are known. Radioisotopes may be detected using gamma, beta or scintillation counters.

Fluorophores that may be used as detectable tags include, for example, resorufin, fluorescein (fluorescein isothiocyanate, FITC), rhodamine (tetramethyl rhodamine isothiocyanate, TRITC), green fluorescent protein (GFP), phycobiliproteins (allophycocyanin, phycocyanin, phycoerythrin and phycoerythrocyanin, derivatives of any of the foregoing) or combinations thereof. In certain embodiments, the detectable tag may be part of the detectable agent (e.g. in the form of a fusion protein or a protein comprising fluorescent amino acids). Fluorophores may be subjected to applied stimulation (for example light of a suitable excitation wavelength) to promote fluorescence.

Luminescent compounds that may be used as detectable tags include, for example, chemiluminescent and/or bioluminescent compounds. These compounds may be used to label the detectable agent. The presence of the chemiluminescent-tag may be determined by detecting the presence of luminescence that arises during the course of a chemical reaction. Examples of useful chemiluminescent labelling compounds are luminol, isoluminol, theromatic acridinium ester, imidazole, acridinium salt and oxalate ester or combinations thereof. Bioluminescence is a type of chemiluminescence found in biological systems in which a catalytic protein increases the efficiency of the chemiluminescent reaction. The presence of a bioluminescent antibody is determined by detecting the presence of luminescence. Examples of bioluminescent compounds include luciferin, luciferase and aequorin.

As described herein, in certain embodiments the method comprises providing to a reaction vessel: a) a sample comprising an analyte; b) a solid substrate comprising a bound immobilisation agent: c) a capture agent comprising a ligand for the immobilisation agent; and d) a detectable agent.

In certain embodiments, the present disclosure provides a method for detecting an analyte in a sample, the method comprising:
providing to a reaction vessel:
   (a) a sample comprising an analyte
   (b) a solid substrate comprising a bound immobilisation agent;
   (c) a capture agent comprising a ligand for the immobilisation agent; and
   (d) a detectable agent:
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

In certain embodiments, the present disclosure provides a method for detecting an analyte in a sample, the method comprising:
providing to a reaction vessel:
   (a) a sample comprising an analyte
   (b) a solid substrate comprising a bound immobilisation agent;
   (c) an antibody capture agent comprising a ligand for the immobilisation agent; and
   (d) a detectable agent:
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

In certain embodiments, the methods and/or kits of the present disclosure comprise contacting the sample, the solid substrate, the capture agent and the detectable agent, in the reaction vessel to form a mixture. In certain embodiments the methods for detecting an analyte comprise contacting the sample, the capture agent, the detectable agent and the solid substrate in the reaction vessel, to form a mixture. In certain embodiments, the components are brought into contact with each other in the reaction vessel, to allow the formation of a complex between the capture agent, the analyte and the detectable agent, the complex being able to be immobilised on the solid substrate (via the ligand on the solid substrate) concurrently and/or after its formation. As described herein, this may provide advantages to the performance of certain methods and/or kits of the present disclosure.

In certain embodiments the methods of the present disclosure comprise contacting the sample, the capture agent, the detectable agent and the solid substrate in the reaction vessel to allow binding of the capture agent and the detectable agent to the analyte to form a complex. Upon formation, the complex may be immobilized on the solid substrate via the ligand binding to the immobilisation agent bound to the solid substrate.

In certain embodiments, the methods and/or kits provide contacting in one or more of at least two reaction vessels, one or more samples, one or more capture agents and one or more detectable agents to allow the formation of one or more complexes comprising an analyte, a capture agent and a detectable agent.

In some embodiments, the methods and/or kits provide contacting one or more complexes with the solid substrate, such that the immobilisation agent may bind the one or more complexes via the ligand.

In certain embodiments, the methods and/or kits of the present disclosure comprise contacting the sample, the capture agent, the detectable agent and the solid substrate in the reaction vessel to allow binding of the capture agent and the detectable agent to the analyte to form a complex.

In certain embodiments, the methods and/or kits of the present disclosure comprise immobilizing the complex on the solid substrate via the ligand binding to the immobilisation agent bound to the solid substrate.

In certain embodiments, the present disclosure provides a method for detecting an analyte in a sample, the method comprising:
providing a reaction vessel;
providing a solid substrate comprising a bound immobilisation agent;
providing a capture agent which can bind the analyte, wherein the capture agent comprises a ligand for the immobilisation agent; and
providing a detectable agent which can bind to the analyte;
contacting the sample, the capture agent, the detectable agent and the solid substrate in the reaction vessel to allow binding of the capture agent and the detectable agent to the analyte to form a complex;
immobilizing the complex on the solid substrate via the ligand binding to the immobilisation agent bound to the solid substrate;
washing the solid substrate in the reaction vessel to remove both the capture agent and the detectable agent not bound to the solid substrate via the ligand; and
detecting the analyte by detecting the presence of the detectable agent present in the complex immobilised to the solid substrate in the reaction vessel.

In certain embodiments, the methods and/or kits of the present disclosure comprise binding the analyte with the capture agent and the detectable agent to form a complex.

In certain embodiments, the methods and/or kits of the present disclosure comprise binding the complex via the ligand with the immobilisation agent to form an immobilised complex.

In certain embodiments, the methods and/or kits comprise removing unbound capture agent and/or unbound detectable agent by washing the solid substrate in the reaction vessel; and detecting the immobilised complex.

In certain embodiments, the present disclosure provides a method for detecting an analyte in a sample, the method comprising:
providing to a reaction vessel:
   (a) a sample comprising an analyte;
   (b) a solid substrate comprising a bound immobilisation agent;
   (c) a capture agent comprising a ligand for the immobilisation agent; and
   (d) a detectable agent;
contacting the sample, the solid substrate, the capture agent and the detectable agent, in the reaction vessel to form a mixture;
binding the analyte with the capture agent and the detectable agent to form a complex;
binding the complex via the ligand with the immobilisation agent to form an immobilised complex;
removing unbound capture agent and/or unbound detectable agent by washing the solid substrate in the reaction vessel; and
detecting the immobilised complex.

In certain embodiments, prior to bringing the components into contact with each other in the reaction vessel, specific individual components may be brought into contact prior with each other.

In certain embodiments, contacting of one or more of the individual components may occur in the reaction vessel or may occur in a separate reaction vessel.

In certain embodiments, the sample, the capture agent, the detectable agent and the solid substrate are not contacted in a separate vessel prior to contacting in the reaction vessel. Thus, in certain embodiments the combination of the components is contacted together for the first time in the reaction vessel.

In certain embodiments, the sample and the solid substrate are contacted prior to contacting with the capture agent and/or the detectable agent. As discussed herein, this may provide in certain embodiments one or more advantages to the performance of some of the methods and/or kits of the present disclosure, as the capture agent and/or the detectable agent are exposed to the analyte in the presence of the solid substrate.

In certain embodiments, the sample (and analyte(s) therein) is exposed to the solid substrate prior to exposure to either or both of the capture agent and the detectable agent. As discussed herein, this may in certain embodiments provide an advantage to the performance of the method, as the capture agent and/or the detectable agent do not come into contact with the analyte until the analyte is in the presence of the solid substrate. For example without being bound by theory, these methods of contacting may provide advantages to the formation of the complex between the capture agent, the analyte and the detectable agent.

In certain embodiments, the sample and the solid substrate are first contacted in the reaction vessel.

In certain embodiments of the present disclosure, wherein the solid substrate forms part of the reaction vessel, the sample may be added to the reaction vessel and subsequently the capture agent and/or the detectable agent are brought into contact with the sample and the solid substrate.

In certain embodiments, the sample and the solid substrate are not substantially incubated prior to contacting with the capture agent and/or the detectable agent. This provides an advantage to the methods of the present disclosure by reducing the time required to detect the analyte.

In certain embodiments, the capture agent and the detectable agent are brought into contact with each other before they are contacted with either or both of the sample and the solid substrate. Typically, this may be achieved by first contacting the capture antibody and the detectable agent in a separate vessel.

In certain embodiments wherein the solid substrate forms part of the reaction vessel, the capture agent and the detectable agent may be brought into contact with each other and then placed in the reaction vessel containing sample.

In certain embodiments, the capture agent and the detectable agent are sequentially contacted with the previously contacted sample and/or the solid substrate.

In certain embodiments, the sample and the solid substrate are brought into contact and then each of the capture agent and the detectable agent are then brought into contact with the sample and the solid substrate. In certain embodiments, the capture agent is first contacted with the sample and the solid substrate and subsequently the detectable agent is brought into contact with the capture agent, the sample and the solid substrate. In certain embodiments, the detectable agent is first contacted with the sample and the solid substrate and subsequently the capture agent is brought into contact with the detectable agent, the sample and the solid substrate.

As described herein, in certain embodiments a complex comprising the analyte, capture agent and detectable agent is formed prior to (and/or concurrently with) binding between the immobilisation agent and the ligand. The complex may be formed by sequential or concurrent addition of the capture agent and detectable agent to the analyte prior to (and/or concurrently with) contacting the complex with the immobilisation agent on the solid substrate.

In certain embodiments, the methods and/kits may utilise a standard ELISA protocol. For example, in certain embodiments, the contacting comprises providing the capture agent and the sample to the reaction vessel, washing the solid substrate and subsequently providing the detectable agent to the reaction vessel.

In certain embodiments, the methods and/or kits of the present disclosure comprise incubating the contacted sample, the solid substrate, the capture agent and the detectable agent.

In certain embodiments, there is an incubation of the contacted sample, the solid substrate, the capture agent and the detectable agent prior to washing of the solid substrate.

In certain embodiments the present disclosure provides a method for detecting an analyte in a sample, the method comprising:
providing a reaction vessel;
providing a solid substrate comprising a bound immobilisation agent;
providing a capture agent which can bind the analyte, wherein the capture agent comprises a ligand for the immobilisation agent;
providing a detectable agent which can bind to the analyte;
contacting the sample, the capture agent, the detectable agent and the solid substrate in the reaction vessel;
incubating the contacted sample, the solid substrate, the capture agent and the detectable agent;
washing the solid substrate in the reaction vessel to remove both the capture agent and the detectable agent not bound to the solid substrate via the ligand; and
detecting the analyte by detecting the presence of the detectable agent bound to the solid substrate in the reaction vessel.

In certain embodiments, the incubating is 2 hours or less, 90 minutes or less, 80 minutes or less, 70 minutes or less, 1 hour or less, 50 minutes or less, 40 minutes or less, 30 minutes or less, 20 minutes or less, 15 minutes or less, 10 minutes or less, or 5 minutes or less. In certain embodiments, if desired, there will be no incubation. In certain embodiments, the incubation is between 10 minutes to 2 hours, 10 minutes to 1 hour, 15 minutes to 2 hours, 15 minutes to 1 hour, 30 minutes to 2 hours, 30 minutes to 1 hour, or 1 hour to 2 hours. In some embodiments, the incubation is at least 5, 10, 15, 20, 25, 30, 60, 70, 80, 90 or 120 minutes.

In certain embodiments the incubation of the contacted sample, the solid substrate, the capture agent and the detectable agent prior to washing of the solid substrate occurs in the reaction vessel.

In certain embodiments, the incubation of the contacted sample, the solid substrate, the capture agent and the detectable agent prior to washing of the solid substrate occurs in a separate vessel.

As described herein, the methods and/or kits for detecting the analyte of the present disclosure comprise washing the solid substrate in the reaction vessel to remove the capture agent and the detectable agent not bound to the solid substrate via the ligand.

The washing of the solid substrate may be performed using a suitable method sufficient to remove capture agent and detectable agent not bound to the solid substrate via the ligand on the capture agent.

In certain embodiments, washing the solid substrate prior to detection of the detectable agent allows the removal of unbound detectable agent and/or detectable agent not bound via the capture agent, which can decrease the level of background signal and hence improve sensitivity. Methods for washing steps are known and generally involve repeated addition and removal of buffer.

In certain embodiments, the solid substrate may be washed one or more times, and with one or more buffers. In certain embodiments, the solid substrate may be washed two or more times, and with one or more buffers. In certain embodiments, the solid substrate may be washed three or more times, and with one or more buffers.

In certain embodiments, there is no additional washing of the solid substrate after contacting of the solid substrate with any one or more of the sample, the capture agent and the detectable agent.

For example, in embodiments utilising a peptide/antibody capture system or a steptavidin/biotin capture system, such systems may assist in reducing the steps involving washing of the solid substrate.

In certain embodiments it will be appreciated that there is no additional washing of the solid substrate after contacting of the solid substrate with any one or more of the sample, the capture agent and the detectable agent. However, in certain embodiments, if desired, additional washing of the solid substrate after contacting of the solid substrate with any one or more of the sample, the capture agent and the detectable agent may be conducted. In certain embodiments, one, two, three or four washings of the solid substrate after contacting of the solid substrate with any one or more of the sample, the capture agent and the detectable agent may be conducted.

In certain embodiments, the methods and/or kits of the present disclosure may be performed using only a single/one wash step conducted during the entire method. The use of a one wash protocol may provide one or more advantages to the method for detecting an analyte. For example, not only does the use of a one wash protocol provide advantages to reducing the number of handling steps involved and the time required to conduct the method, the one wash protocol may also provide an improvement in the efficiency and performance of the method.

In certain embodiments, a streptavidin/biotin capture system assists in the use of single wash protocol.

However, in certain embodiments, the one wash protocol may be varied, if desired, to add additional quick washes or rinses at various stages of the protocol or to add addition washes or rinses at various stages of the protocol.

In certain embodiments, there is no additional washing of the solid substrate after contacting of the solid substrate with any one or more of the sample, the capture agent and the detectable agent.

In certain embodiments, the present disclosure provides a method for detecting an analyte in a sample, the method comprising:
providing a reaction vessel;
providing a solid substrate comprising a bound immobilisation agent;
providing a capture agent which can bind the analyte, wherein the capture agent comprises a ligand for the immobilisation agent;
providing a detectable agent which can bind to the analyte;
contacting the sample, the capture agent, the detectable agent and the solid substrate in the reaction vessel;
washing the solid substrate in the reaction vessel to remove both the capture agent and the detectable agent not bound to the solid substrate via the ligand, wherein there is no additional washing of the solid substrate after contacting of the solid substrate with any one or more of the sample, the capture agent and the detectable agent; and
detecting the analyte by detecting the presence of the detectable agent bound to the solid substrate in the reaction vessel.

As described previously herein, the methods and/or kits of the present disclosure comprise detecting the analyte by detecting the presence of the detectable agent bound to the solid substrate in the reaction vessel.

In this regard, in certain embodiments the detection of the analyte is achieved by detecting the presence of the detectable agent bound to the solid substrate. The detectable agent may be detected while bound to the solid substrate and/or after release from the solid substrate.

In certain embodiments, the detection of the analyte is achieved by detecting a complex immobilised to the solid substrate in the reaction vessel.

In certain embodiments, the methods and/or kits comprise detecting the presence of one or more immobilised complexes on the solid substrate by detection of one or more detectable agents.

In certain embodiments, the methods and/or kits of the present disclosure comprise detecting the analyte by detecting the presence of the detectable agent present in the complex immobilised to the solid substrate in the reaction vessel.

The detection of the analyte by detecting the presence of the detectable agent bound to the substrate may be achieved by a suitable method specific to the detectable agent. Examples of detectable agents are as hereinbefore described.

As described herein, the time taken to perform a method for detecting an analyte in a sample is an important consideration.

For example, ELISA can be time-intensive and it is not uncommon in ELISA for incubation steps to be performed after the addition of each individual component of the ELISA.

As discussed herein, in certain embodiments the present disclosure minimises one or more of the number of incubation, handling and/or washing steps. In some embodiments, this makes the method of the present disclosure particularly amenable to automation. Previous assays are difficult to automate as multiple handling steps are needed, including several aspiration, dispensing, and washing steps.

In certain embodiments, reducing the number of incubation steps and/or washing steps that are required may allow the duration of the complex to solid substrate binding step to be maximised without increasing the total duration of the method. Increasing the duration of the complex to solid substrate binding step may in certain embodiments increase the sensitivity of the method.

Certain embodiments of the present disclosure contemplate, if desired, various combinations as to the number of incubation, handling and/or washing steps.

As described herein, in certain embodiments the detection of the analyte is achieved in a time of 2 hours or less. In certain embodiments, the detection of the analyte is achieved in a time of 2 hours or less from contacting the sample with the capture agent and/or the detectable agent. Examples of times for achieving detection of the analyte are as described herein.

In certain embodiments, the detection of the analyte is achieved in a time of 120 minutes or less. In certain embodiments, the detection of the analyte is achieved in a time of 120 minutes or from contacting the sample with the capture agent and/or the detectable agent. In certain embodiments, the detection of the analyte is achieved in a time of 60 minutes or less. In certain embodiments, the detection of the analyte is achieved in a time of 60 minutes or less from contacting the sample with the capture agent and/or the detectable agent. In certain embodiments, the detection of the analyte is achieved in a time of 30 minutes or less. In certain embodiments, the detection of the analyte is achieved in a time of 30 minutes or less from contacting the sample with the capture agent and/or the detectable agent. In certain embodiments, the detection of the analyte is achieved in a time of 15 minutes or less. In certain embodiments, the detection of the analyte is achieved in a time of 15 minutes or less from contacting the sample with the capture agent and/or the detectable agent. In certain embodiments, the detection of the analyte is achieved in a time of 10 minutes or less. In certain embodiments, the detection of the analyte is achieved in a time of 10 minutes or less minutes from contacting the sample with the capture agent and/or the detectable agent.

As described herein, in certain embodiments the use of a capture system may assist in reducing the time required to undertake an assay. For example, in embodiments utilising a peptide/antibody capture system or a steptavidin/biotin capture system, such systems may assist in reducing the time to undertake such an assay.

As described herein, in certain embodiments the use of a capture system may assist in reducing the washing of the solid substrate and reducing the time required to undertake an assay. For example, in embodiments utilising a peptide/antibody capture system or a steptavidin/biotin capture system, such systems may assist in reducing the washing of the solid substrate and in reducing the time to undertake such an assay.

In certain embodiments the methods and/or kits shows a low variability for detecting an analyte between reactions. In certain embodiments, the methods and/or kits show a low intra-plate variability. In certain embodiments, the intra-plate variability is 30% or less, 20% or less, or 10% or less. For example, in certain embodiments the methods and/or kits show a low intra-plate variability for detecting an analyte, such as an intra-plate variability of 30% or less, 20% or less, or 10% or less.

In certain embodiments, a further advantage of some of the methods of the present disclosure is the ability to use a single assay plate or platform that is suitable for many different assay kits. This may provide manufacturers with a number of benefits, including reduced cost, labor and quality control requirements, in comparison to preparing a different assay plate for every assay kit, as is the current standard for ELISA kit manufacture. In addition, in certain embodiments inputs can be reduced by the ability to use less of the target-specific capture antibody, again reducing costs and quality control requirements, as single batches of target-specific antibodies can be used for more assay kits.

In certain embodiments, the present disclosure provides a method for detecting one or more analytes in one or more samples using a single assay platform, the method comprising:
providing one or more samples comprising one or more analytes to be detected;
providing an assay platform comprising a plurality of reaction vessels, one or more of the plurality of reaction vessels comprising the same bound immobilisation agent;
providing one or more capture agents, the one or more capture agents being able to bind to the one or more analytes to be detected and comprising a ligand for the immobilisation agent;
providing one or more detectable agents, the one or more detectable agents being able to bind to the one or more analytes to be detected;
contacting the one or more samples, the one or more capture agents and the one or more detectable agents in one or more of the plurality of reaction vessels in the assay platform;
washing one or more of the plurality of reaction vessels to remove the one or more capture agents and the one or more detectable agents not bound via the ligand in one or more of the plurality of reaction vessels; and
detecting the one or more analytes in one or more of the plurality of reaction vessels by detecting the presence of the one or more detectable agents bound to one or more of the plurality of reaction vessels.

In certain embodiments, the present disclosure provides a method for detecting one or more analytes in one or more samples using a single assay platform, the method comprising:
providing one or more samples comprising one or more analytes to be detected;
providing an assay platform comprising a plurality of reaction vessels, one or more of the plurality of reaction vessels comprising the same bound immobilisation agent;
providing one or more antibody capture agents in solution, the one or more antibody capture agents being able to bind to the one or more analytes to be detected and comprising a ligand for the immobilisation agent;
providing one or more detectable agents in solution, the one or more detectable agents being able to bind to the one or more analytes to be detected;
contacting the one or more samples, the one or more capture agents and the one or more detectable agents in one or more of the plurality of reaction vessels in the assay platform;
washing one or more of the plurality of reaction vessels to remove the one or more capture agents and the one or more detectable agents not bound via the ligand in one or more of the plurality of reaction vessels; and
detecting the one or more analytes in one or more of the plurality of reaction vessels by detecting the presence of the one or more detectable agents bound to one or more of the plurality of reaction vessels.

In certain embodiments, the present disclosure provides a method for detecting one or more analytes in one or more samples using a single assay platform, the method comprising:
providing one or more samples comprising one or more analytes to be detected;
providing an assay platform comprising at least two reaction vessels, the at least two reaction vessels comprising the same bound immobilisation agent;
providing one or more capture agents, the one or more capture agents being able to bind to the one or more analytes to be detected and comprising a ligand for the immobilisation agent;
providing one or more detectable agents, the one or more detectable agents being able to bind to the one or more analytes to be detected;
contacting the one or more samples, the one or more capture agents and the one or more detectable agents in one or more of the at least two reaction vessels in the assay platform;
washing one or more of the at least two reaction vessels to remove the one or more capture agents and the one or more detectable agents not bound via the ligand in one or more of the at least two reaction vessels; and
detecting the one or more analytes in one or more of the at least two reaction vessels by detecting the presence of the one or more detectable agents bound to one or more of the at least two reaction vessels.

In certain embodiments, the present disclosure provides a method for detecting one or more analytes in one or more samples using a single assay platform, the method comprising:
providing one or more samples comprising one or more analytes to be detected;
providing an assay platform comprising at least two reaction vessels, the at least two reaction vessels comprising the same bound immobilisation agent;
providing one or more antibody capture agents in solution, the one or more antibody capture agents being able to bind to the one or more analytes to be detected and comprising a ligand for the immobilisation agent;
providing one or more detectable agents in solution, the one or more detectable agents being able to bind to the one or more analytes to be detected;
contacting the one or more samples, the one or more capture agents and the one or more detectable agents in one or more of the at least two reaction vessels in the assay platform;
washing one or more of the at least two reaction vessels to remove the one or more capture agents and the one or more detectable agents not bound via the ligand in one or more of the at least two reaction vessels; and
detecting the one or more analytes in one or more of the at least two reaction vessels by detecting the presence of the one or more detectable agents bound to one or more of the at least two reaction vessels.

In certain embodiments, the present disclosure provides a kit for performing the methods as described herein. Kits are as described herein.

In certain embodiments, the assay platform is a multi-well plate, such as a microtitre plate. Other types of assay platform are contemplated. Assay platforms are as described herein.

In certain embodiments, the immobilisation agent comprises an antibody and/or an antigen binding part thereof. Other types of immobilisation agents are contemplated. Immobilisation agents, and their respective binding partners, are as described herein.

In certain embodiments, the ligand is a peptide tag. Other types of ligands are contemplated. Ligands, and their respective binding partners, are as described herein.

In certain embodiments, the immobilisation agent is an anti-peptide tag antibody.

In certain embodiments, the detectable agent comprises an antibody. Other types of detectable agents are contemplated. Detectable agents are as described herein.

In certain embodiments, the detectable agent comprises a detectable tag. In certain embodiments, the detectable tag comprises one or more of an enzyme, a fluorophore, and a lanthanide ion. Other types of detectable agents are contemplated. Detectable agents are as described herein.

In certain embodiments of the methods and/or kits, more than one analyte may be detected in one reaction vessel. In certain embodiments, a plurality of analytes is detected. In certain embodiments, the methods and/or kits comprise multiplex detection of a plurality of analytes.

In certain embodiments, more than one analyte may be detected in one reaction vessel. In certain embodiments, one analyte is detected in a sample. In some embodiments, one or more analytes are detected in a sample. In certain embodiments, at least two analytes are detected in a sample.

In certain embodiments the detection of more than one analyte may be achieved by providing several target-specific antibody capture agents to the reaction vessel, in combination with providing their respective detection agents. For example, in certain embodiments where each detection agent is an antibody, each antibody may be conjugated to a different detectable tag, such as an enzyme, a fluorophore, a lanthanide, a chelate or combinations thereof.

In certain embodiments, at least two different analytes are detected. In certain embodiments, a plurality of different analytes is detected in the reaction vessel.

In certain embodiments, the method comprises at least two different capture agents that bind to the at least two analytes.

In certain embodiments, the solid substrate comprises a single immobilisation agent. In certain embodiments, the solid substrate comprises at least two different immobilisation agents.

In certain embodiments, the method comprises at least two detectable agents. In certain embodiments, the at least two detectable agents comprise one or more detectable tags. In certain embodiments, the one or more detectable tags comprise a fluorophore.

In certain embodiments, the one or more detectable tags comprise one or more lanthanide ions. In certain embodiments, the lanthanide ion is one or more of Eu3+, Sm³⁺, Tb³⁺, and Dy³⁺. In certain embodiments, one of the detectable tags comprises Eu³⁺ and another of the detectable tags comprises Sm³⁺.

In certain embodiments, one or more of the detectable tags comprises an enzyme that converts a substrate into a detectable product. In certain embodiments, the enzyme is selected from the group consisting of horse radish peroxidase, alkaline phosphatase, and beta-galactosidase.

As described herein, certain embodiments also allow detectable signals to be produced with less capture agent and/or reduced solid substrate surface area relative to ELISA, and as such some methods may be suitable for microfluidic systems, where miniaturisation of structures and minimisation of reagents used is desirable. Accordingly, in certain embodiments, the method may be performed in a microfluidic system. Examples of microfluidic systems may include, for example, microfluidic “lab-on-a-chip” type devices; high density microtitre plates, such as 1536, 3456 or 9600 well microtitre plates; microarrays and the like.

In certain embodiments, the present disclosure provides a kit for detecting an analyte in a sample. In certain embodiments, the present disclosure provides a kit for detecting an analyte using a method as described herein.

In certain embodiments, the methods and/or kits of the present disclosure may also be performed by utilising reagents and/or instructions.

In certain embodiments, a kit(s) is utilised for performing the method(s) of the present disclosure. The kit(s) may comprise one or more of the reagents herein described and/or instructions to assist in the performance of the method.

In certain embodiments, the kit comprises an assay platform comprising a plurality of reaction vessels, one or more of the reaction vessels comprising a bound immobilisation agent. In certain embodiments, the bound immobilisation agents are the same. In certain embodiments, the bound immobilisation agents are different.

In certain embodiments, the assay platform comprises a multi-well plate. Assay platforms are as described herein.

In certain embodiments, the assay platform comprises a plurality of reaction vessels. In certain embodiments, one or more of the reaction vessels comprise a bound immobilisation agent. In certain embodiments, the bound immobilisation agent comprises an antibody to a peptide tag. Immobilisation agents are as described herein.

In certain embodiments, the kit comprises a capture agent which can bind to the analyte, the capture agent comprising a ligand for the immobilisation agent. In certain embodiments, the capture agent comprises an antibody. Capture agents are as described herein. In certain embodiments, the ligand comprises a peptide tag. Ligands are as described herein. In certain embodiments, the capture agent comprises a plurality of ligands. In certain embodiments, the capture agent is in solution.

In certain embodiments, the kit comprises a detectable agent which can bind to the analyte. In certain embodiments, the detectable agent comprises an antibody and/or an antigen binding part thereof. Detectable agents are as described herein. In certain embodiments, the detectable agent is in solution.

In certain embodiments, the detectable agent comprises a detectable tag. In certain embodiments, the detectable tag comprises one or more of an enzyme, a fluorophore, and a lanthanide ion. Detectable tags are as described herein.

In certain embodiments, the kit comprises a composition comprising the capture agent and the detectable agent. In certain embodiments, the composition is a liquid composition.

In certain embodiments, the kit comprises one or more solutions for washing the solid substrate. In certain embodiments, the kit comprises one or more reagents for detecting the detectable agent.

In certain embodiments, the present disclosure provides a kit for detecting an analyte, the kit comprising:
an assay platform comprising a plurality of reaction vessels, one or more of the reaction vessels comprising a bound immobilisation agent;
a capture agent which can bind to the analyte, wherein the capture agent comprises a ligand for the immobilisation agent;
a detectable agent which can bind to the analyte; and
optionally one or more solutions for washing the solid substrate and/or instructions for detecting the analyte.

In certain embodiments, the present disclosure provides a kit for detecting an analyte, the kit comprising:
an assay platform comprising a plurality of reaction vessels, one or more of the reaction vessels comprising a bound anti-peptide tag antibody;
an antibody capture agent in solution which can bind to the analyte, wherein the capture agent comprises a plurality of peptide tags;
an antibody detectable agent in solution which can bind to the analyte, wherein the antibody detectable agent comprises one or more of an enzyme, a fluorophore and a lanthanide ion; and
optionally one or more solutions for washing the solid substrate and/or instructions for detecting the analyte.

In certain embodiments, kit comprises instructions for detecting the analyte comprise instructions for detecting the analyte in a time of 2 hours or less, 90 minutes or less, 80 minutes or less, 70 minutes or less. In certain embodiments, the instructions for detecting the analyte comprise instructions for detecting the analyte in a time of 1 hour or less, 45 minutes or less, 30 minutes or less, 20 minutes or less, 15 minutes or less, 10 minutes or less. In certain embodiments, the kit comprises instructions for the detecting of the analyte is a time period of between 10 minutes to 120 minutes, 10 minutes to 90 minutes, 10 minutes to 60 minutes, 10 minutes to 30 minutes, 15 minutes to 120 minutes, 15 minutes to 90 minutes, 15 minutes to 60 minutes, 30 minutes to 120 minutes, 30 minutes to 90 minutes, 30 minutes to 60 minutes, 45 minutes to 120 minutes, 45 to 90 minutes, 45 minutes to 75 minutes, or 45 minutes to 60 minutes.

In certain embodiments, the instructions for detecting the analyte comprise instructions for utilising only a single wash of the solid substrate after contacting of the solid substrate with any one or more of the sample, the capture agent and the detectable agent.

Certain embodiments provide a kit for detecting an analyte, the kit comprising:
an antibody capture agent which can bind to an analyte, wherein the capture agent comprises a ligand for an immobilisation agent bound to a solid substrate;
a detectable agent which can bind to the analyte; and
instructions for detecting the analyte by contacting the sample, the capture agent, the detectable agent and the solid substrate in a reaction vessel, the detection of the analyte further utilising only a single wash of the solid substrate after contacting of the solid substrate with any one or more of the sample, the capture agent and the detectable agent.

Certain embodiments provide a kit for detecting an analyte, the kit comprising:
an antibody capture agent which can bind to an analyte, wherein the capture agent comprises a ligand for an immobilisation agent bound to a solid substrate;
a detectable agent which can bind to the analyte; and
instructions for detecting the analyte in a time of 2 hours or less from contacting the sample with the capture agent and/or the detectable agent.

As described herein, in certain embodiments, a further advantage of some of the methods of the present disclosure is the ability to use a single assay plate or platform that is suitable for many different assay kits. This may provide manufacturers with a number of benefits, including reduced cost, labor and quality control requirements, in comparison to preparing a different assay plate for every assay kit, as is the current standard for ELISA kit manufacture. In addition, in certain embodiments inputs can be reduced by the ability to use less of the target-specific capture antibody, again reducing costs and quality control requirements, as single batches of target-specific antibodies can be used for more assay kits.

The present disclosure is further described by the following examples. It is to be understood that the following description is for the purpose of describing particular embodiments only and is not intended to be limiting with respect to the above description.

### Example 1

### Materials

Antibodies used in the following examples include: anti-pERK mouse monoclonal (+/− biotinylation); anti-total ERK rabbit monoclonal (+/− HRP); donkey anti-rabbit-HRP conjugate; anti-S6 p240/44 rabbit polyclonal (HRP conjugated); anti-S6 mouse monoclonal (biotinylated); anti-AKT pT308 rabbit monoclonal (HRP conjugated); anti-AKT mouse monoclonal (biotinylated); anti-AKT pS473 mouse monoclonal (biotinylated); and anti-AKT rabbit monoclonal (HRP conjugated).

Other reagents and materials used in the following examples include: QuantaRed™ enhanced chemifluorescent HRP substrate (Thermo Scientific); SIGMAFAST™ OPD tablets (Sigma); 96 well clear immunoassay Maxisorp™ plates (Nunc); 384 well clear immunoassay Maxisorp™ plates (Nunc); Streptavidin (Sigma); Blocking solution (1% BSA in PBS containing 0.05% Tween 20); and A431 cell lysate containing pERK.

### Example 2

### Methods

### 1-Wash ELISA Protocol

Nunc 96 well Maxisorp™ plates were passively coated with streptavidin and blocked. pERK cell lysates (50 μL) were added to wells followed by the addition of a reaction buffer (50 μL) containing pre-optimised concentrations of biotinylated anti-pERK mouse mAb and anti-total ERK-HRP rabbit mAb (alternatively a reaction buffer containing biotinylated anti-pERK mouse mAb, anti-total ERK rabbit mAb and anti-rabbit IgG-HRP can be used).

In certain cases a pre-incubation of pERK cell lysate with the antibodies was performed in a sample plate prior to transfer to the streptavidin coated plate. Plates were incubated for a minimum of 30 min before washing 3× with PBS-T, addition of HRP substrate (100 μL) and measurement of product. A similar 1-wash protocol was followed when using Nunc 384 well Maxisorp™ plates. The specific kinase antibodies were supplemented into the protocol and the final reaction volume was 20 μL.

### Comparative Multi-Wash ELISA Protocol—Streptavidin Coated Plate

Nunc 96 well Maxisorp™ plates were passively coated with streptavidin and blocked. Biotinylated anti-pERK mouse mAb was added to wells and incubated for a minimum of 30 min (100 μL). Plates were washed 3× with PBS-T. pERK cell lysates were added to wells and incubated for a minimum of 30 min (100 μL). Plates were washed 3× with PBS-T. Anti-total ERK-HRP rabbit mAb was added to wells and incubated for a minimum of 30 min (100 μL). Plates were washed 3× with PBS-T before addition of HRP substrate (100 μL) and measurement of product.

### Comparative Multi-Wash ELISA Protocol—Anti-pERK IgG Coated Plate

Nunc 96 well Maxisorp™ plates were passively coated with anti-pERK mouse mAb and blocked. pERK cell lysates were added to wells and incubated for a minimum of 30 min (100 μL). Plates were washed 3× with PBS-T. Anti-total ERK-HRP rabbit mAb was added to wells and incubated for a minimum of 30 min (100 μL). Plates were washed 3× with PBS-T before addition of HRP substrate (100 μL) and measurement of product.

### Example 3

### Results—Assay Characteristics

### Speed/Simplicity

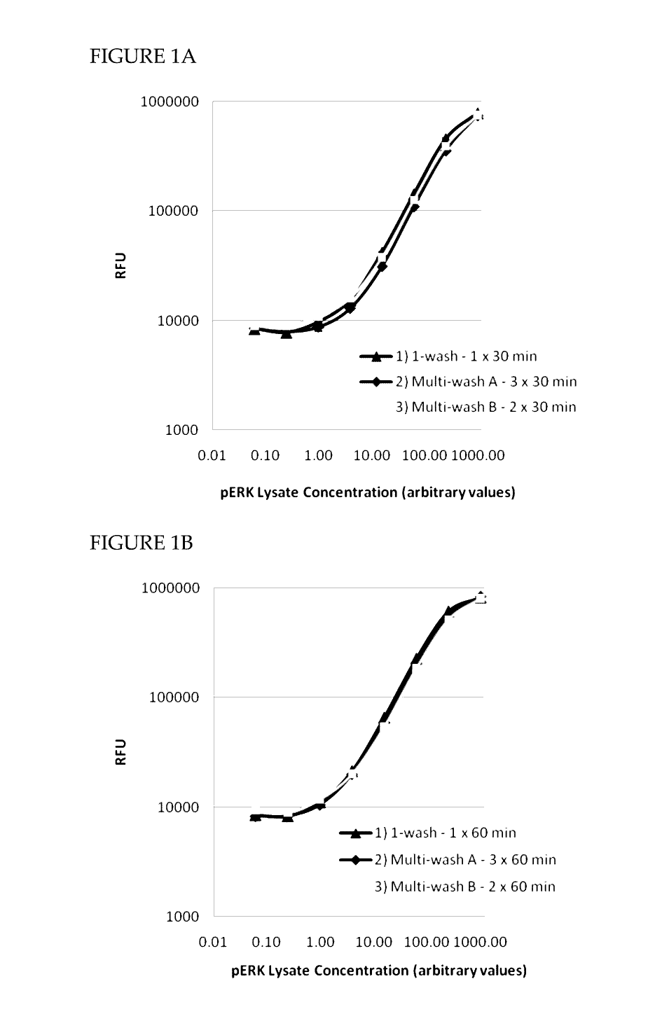

In their optimized formats, the 1-wash assay performed comparably to the multi-wash assay in terms of sensitivity (FIG. 1). This was evident on both streptavidin (Protocols 1 & 2) and anti-pERK IgG (Protocol 3) coated plates for the 30 min (FIG. 1A) and 60 min incubation periods (FIG. 1B). Generally, there was approximately a 10% greater signal obtained at each respective pERK concentration in the 1-wash ELISA compared to the multi-wash ELISAs but this did not translate to a significant improvement in the assay detection limit. Importantly, this demonstrated that the 1-wash assay could be performed with less handling steps and in less than half the time of the multi-wash ELISAs without negatively impacting on sensitivity. This translated to a much simpler ELISA assay format by the consolidation of multiple steps into a single 1-wash/step system.

### Sensitivity

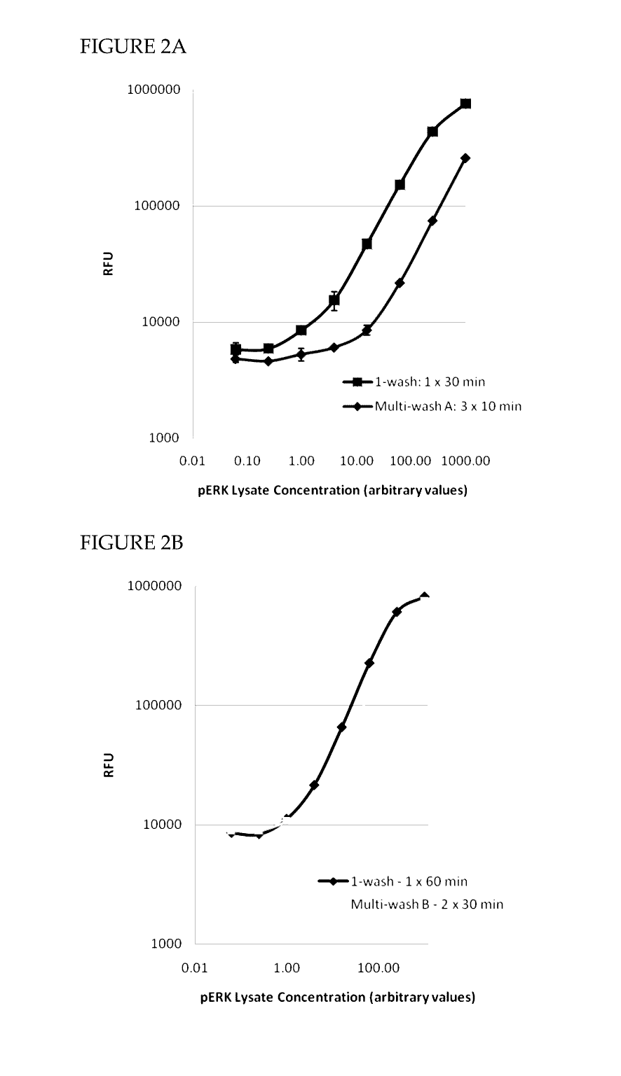

When the 1-wash and multi-wash ELISAs were performed for the same total length of time of 1 h or less, the 1-wash ELISA was superior in sensitivity (FIG. 2). Comparison of a 1×30 min incubation step to 3×10 min incubation steps on a streptavidin coated plate (2A) showed that the 1-wash system was approximately 10 times more sensitive than the multi-wash system. Although not as significant, this trend was also noticeable when comparing a 1×60 min 1-wash assay system on a streptavidin plate, to a 2×30 min multiwash system on an anti-pERK IgG coated plate (2B). The major benefit of the 1-wash ELISA protocol was that it allowed multiple antibody-antigen binding events to occur simultaneously in the single 30 or 60 minute incubation period thereby improving the pERK detection capabilities per unit time.

### Capture Antibody Efficiency

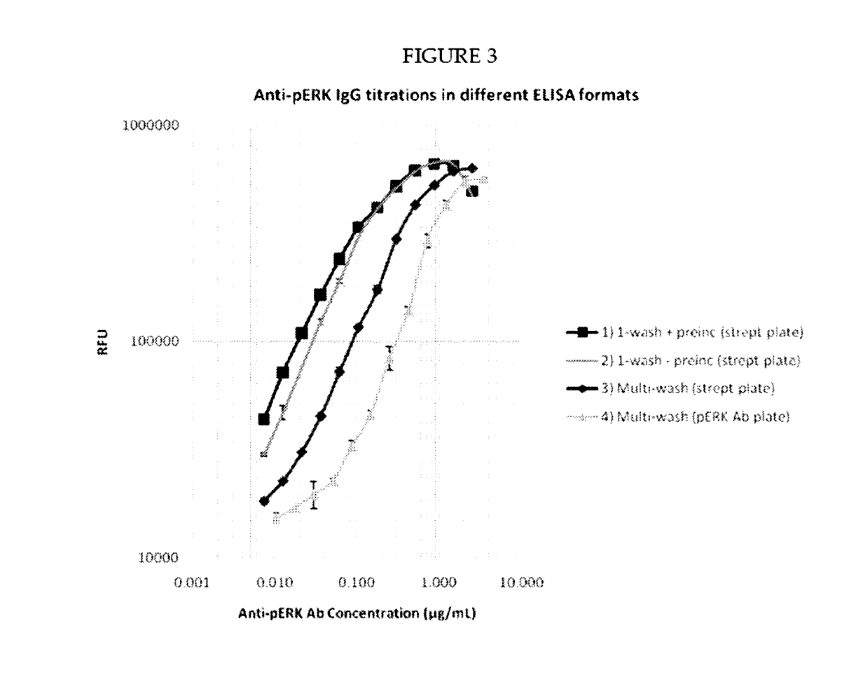

The concentration dependency of anti-pERK IgG (+/− biotinylation) for detecting pERK was assessed in each of the ELISA protocols (FIG. 3). With or without a pre-incubation step, the 1-wash protocol required approximately 4× and 10× less anti-pERK IgG, to detect the same amount of pERK when compared to multi-wash ELISA protocols 3 and 4 respectively. The importance of a pre-incubation step (protocol 1 vs protocol 2) in the 1-wash ELISA was noticeable when the anti-pERK IgG concentrations were 100 ng/mL or less. At these lower concentrations, more pERK per unit antibody (approx 15% higher signal) was able to be detected when a pre-incubation step was incorporated into the 1-wash protocol. Collectively, these results indicated that the 1-wash protocol was more efficient with its use of anti-pERK IgG compared to the multi-wash format for detecting the same amount of pERK. A possible explanation for this phenomenon was that the 1-wash format allowed the formation of solution-phase pERK immune complexes, enabling their binding to the streptavidin or anti-pERK IgG coated surface in a more orientated fashion thereby enhancing antibody functionality. Conversely, in the absence of pERK and detection IgG, biotinylated or unbiotinylated anti-pERK IgG could bind randomly to the surface, which may have led to a portion of pERK IgG binding sites becoming inaccessible to pERK and/or sterically hindering subsequent binding events in the sandwich (i.e. detection IgG).

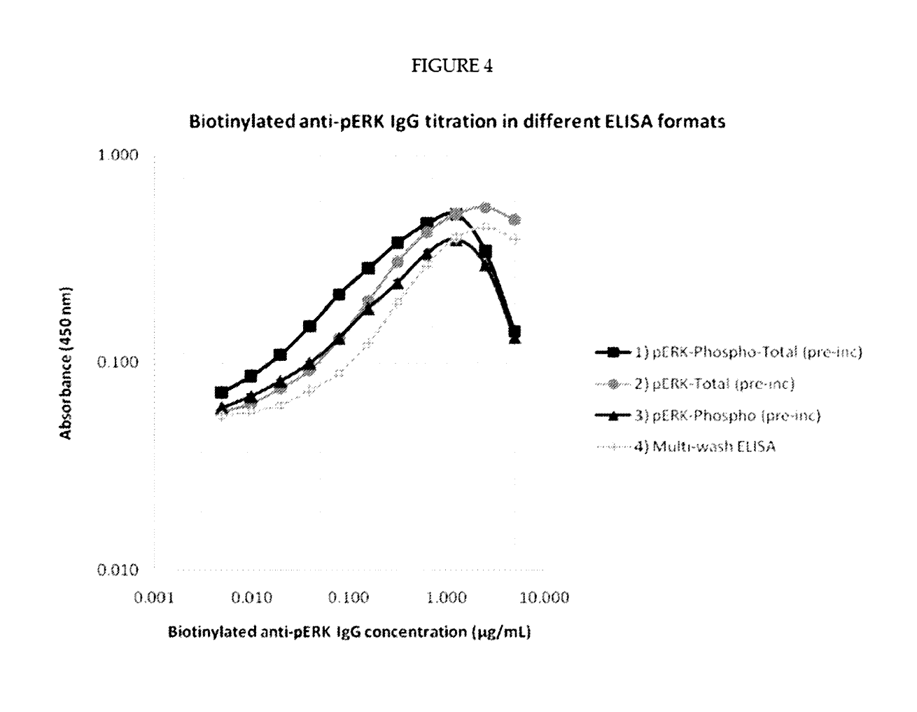

The improved anti-pERK IgG efficiency phenomenon highlighted in FIG. 3 for the 1-wash ELISA format was investigated further by separating the multiple antibody-antigen binding events of the pERK assay (FIG. 4). This highlighted that independent formation of pERK with anti-total ERK-HRP IgG or anti-pERK IgG (protocols 2 & 3 respectively), prior to binding to their immobilized partner on the plate, contributed to the more efficient use of anti-pERK IgG in the 1-wash ELISA format. Individually, protocols 2 & 3 were approximately 2 times more efficient with their use of anti-pERK IgG for detecting pERK compared to the multi-wash ELISA (protocol 4). Furthermore when the individual binding events of protocols 2 & 3 were allowed to occur simultaneously as part of the 1-wash ELISA (protocol 1), the use of anti-pERK IgG compared to the multi-wash procedure was 4-5 times less when measuring the same concentration of pERK. Ultimately this highlighted that the binding of both antibodies to pERK in solution were important for enhancing the functionality of the anti-pERK IgG used in the 1-wash ELISA. This would result in less reagent use (i.e. antibody) and therefore reduced assay cost, compared to the multi-wash ELISA format.

### Versatility

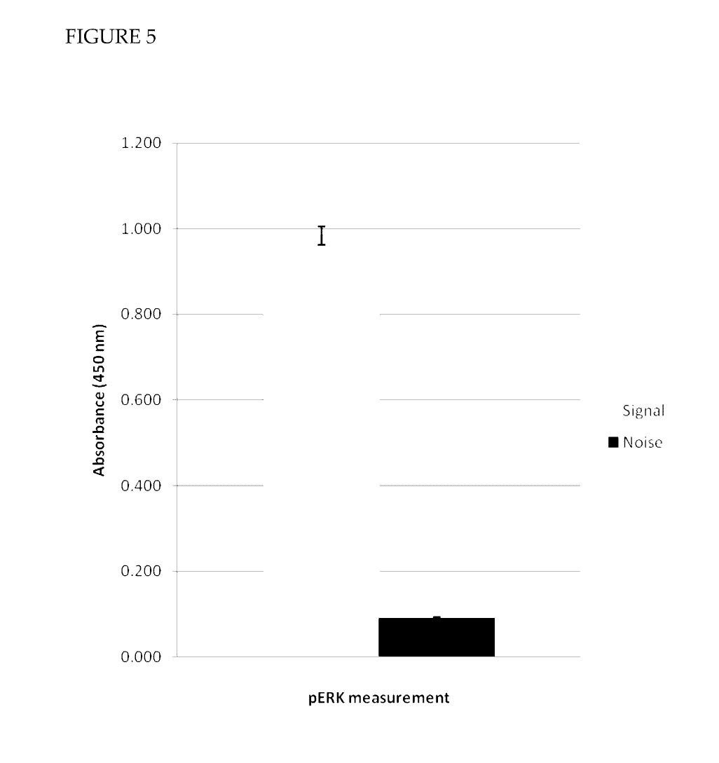

The 1-wash ELISA protocol was also challenged using a secondary detection antibody that was conjugated to HRP (FIG. 5). This was achieved by replacing the anti-total ERK-HRP with the original unconjugated antibody (i.e. minus HRP) and introducing anti-rabbit IgG-HRP as the secondary detection antibody. That is, this experiment used a 3 antibody protocol in the 1-wash ELISA format and yielded an A450 signal for pERK of approximately 1.0 AU and a signal:noise value of 10. Although unoptimized, in principle this secondary detection approach was validated in a 1-wash protocol and highlighted the versatility of the 1-wash ELISA using at least 3 antibodies.

### Robustness

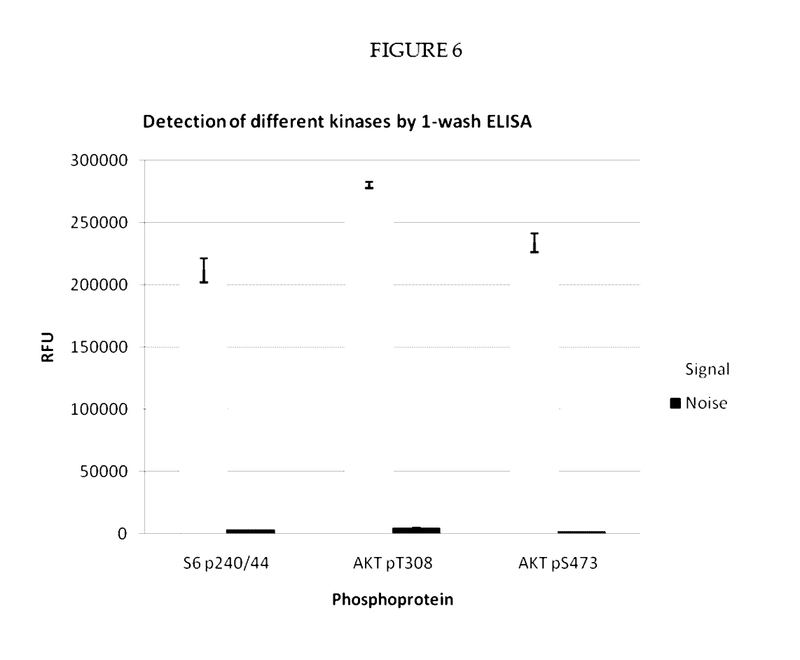

Detection of other phosphoproteins including S6 p240/44, AKT pT308 and AKT pS473 was also achieved in the 1-wash ELISA system (FIG. 6). In 384 well streptavidin coated plates, signal:noise ratios of greater than 60 were achieved when assaying cell lysates containing the specific phosphoproteins of interest. Like the pERK protocol, the AKT pS473 assay also used an anti-phospho IgG as the capture antibody with an anti-total IgG used as the detection antibody (i.e. conjugated to HRP). Alternatively the S6 p240/44 and AKT pS473 assays used an anti-total IgG as the capture antibody, with a specific anti-phospho IgG-HRP completing the sandwich. These results demonstrated the robustness of the 1-wash ELISA with its ability to detect different targets in varying immune complex orientations.

### Example 4

### Microfluidics

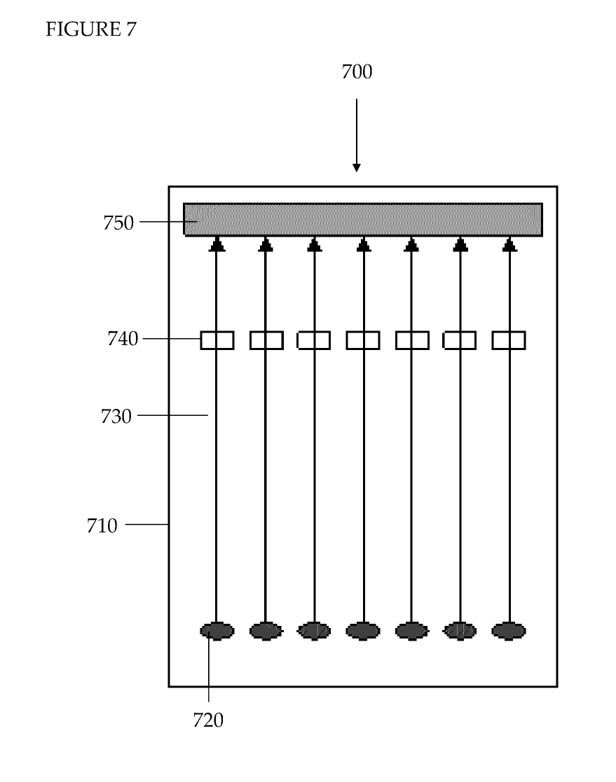

Microfluidic reactions were performed in a microfluidic cartridge as shown in FIG. 7. Referring to FIG. 7, the microfluidic cartridge 700 comprises a plastic substrate 710 into which a plurality of flow channels 730 are formed. A sample is introduced into the flow channel 730 via sample inlet 720. The sample is then driven along flow channel 730 by a pump (not shown). Detection region 740 comprises an electrode for electrochemical detection to which an immobilization agent is bound. In the embodiments described in the following examples, the immobilization agent is streptavidin. Moreover, although the present invention contemplates any suitable electrodes and methods for electrochemical detection, the method described in the following examples utilizes the electrodes and detection methods described in U.S. Pat. No. 6,770,190. After passing over detection area 740, the sample is transported to waste collection area 750.

An example of the method of the present invention performed in the microfluidic cartridge is described below:

Samples were mixed with a reaction buffer (phosphate buffered saline, BSA 0.3%, Tween 0.1%) containing two antibodies to the analyte of interest. For each analyte, the two antibodies were raised against distinct epitopes on the analyte of interest, such that both antibodies could bind to the protein of interest simultaneously. One of the antibodies performed the function of a capture agent and had biotin attached to it, while the other antibody performed the function of a detectable agent and was linked to horse radish peroxidise (HRP).

The samples being measured contained varying amounts of an analyte of interest, in the present examples either phospho-ERK or phospho-AKT. A microfluidic cartridge (see FIG. 7) was placed on a pumping and detection instrument, and samples were drawn onto the microfluidic cartridge into separate lanes of the cartridge. The cartridge bound the biotinylated antibody at the detection region. As set out above, the detection region comprised an electrode for electrochemical detection to which streptavidin is bound as an immobilisation agent. As such, a complex comprising biotinylated capture antibody, bound analyte and HRP-linked detectable antibody would become immobilised to the electrode via interaction of the biotin on the capture antibody and streptavidin on the electrode.

After capture, the cartridge was automatically washed with buffer without antibodies. Following this wash step, a solution containing HRP substrate (SigmaFAST OPD) was drawn over the cartridge, allowing bound HRP to convert the HRP substrate to products that could be detected electrochemically by the electrode and detection equipment present on the pumping device. The electrical signals generated were proportional to the level of HRP-induced product conversion, which was proportional to the amount of analyte bound to the capture antibodies.

### Example 5

Detection of pERK Using a Microfluidic System

Recombinant pERK was diluted in 1× lysis buffer, with four fold dilutions from a top concentration of 400 ng/ml (10 nM). Samples were pre incubated with an equal volume of reaction buffer (see above).

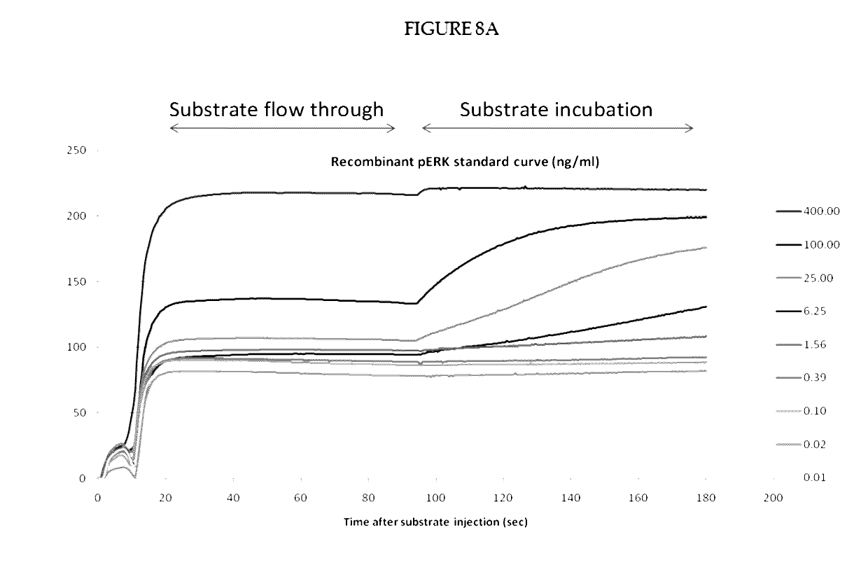

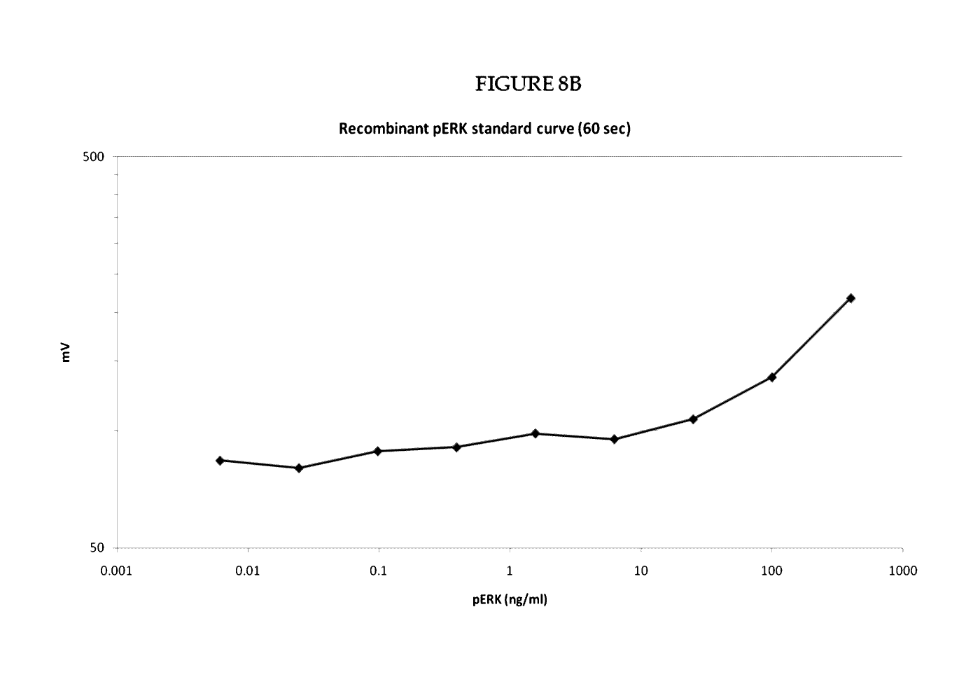

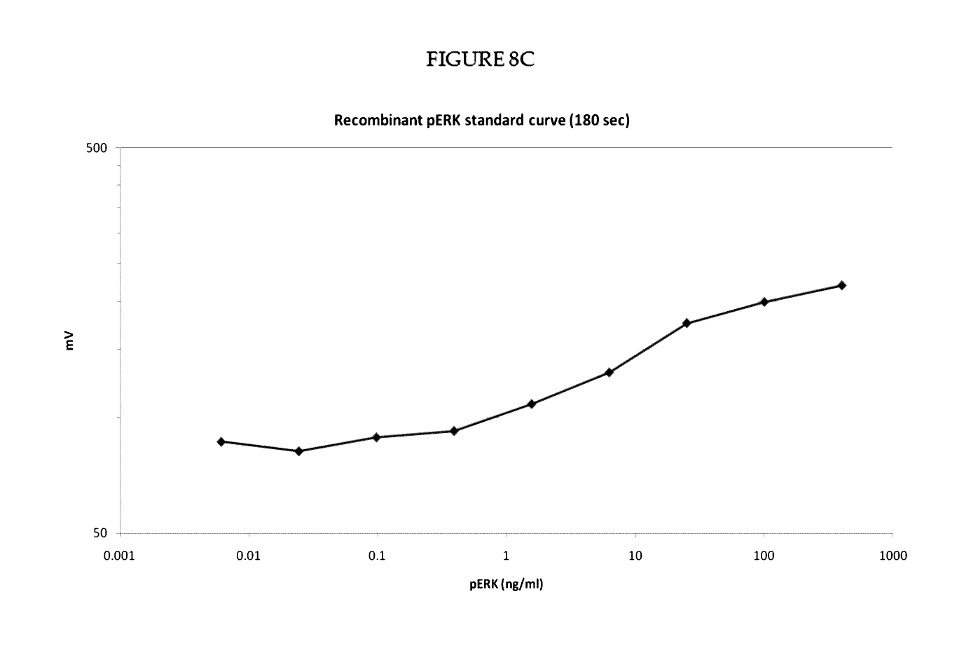

The sample/reaction buffer mix was then run on a microfluidic cartridge as described in Example 4. The results are shown in FIGS. 8A-8C. Each data point shown is the average of 3 flow cells from a single cartridge. The data was transformed by taking the point at which substrate injection begins as zero. Data was collected from the point at which substrate flow through begins up until the end of substrate incubation phase (180 s after substrate injection).

As can be seen by comparing FIGS. 8b and 8c, data collection at the end of the substrate incubation phase (180 s after substrate injection) appeared to provide greater sensitivity. Using the data taken from 180 seconds after injection of substrate, the detection limit of the chip was about 2 ng/ml pERK.

### Example 6

Detection of pAKT Using a Microfluidic System

Recombinant pAKT473 was diluted in 1× lysis buffer, with five fold dilutions from a top concentration of 100 ng/ml. Samples were pre incubated for two hours with an equal volume of reaction buffer (see above) to equilibrate the interaction and so minimise incubation effects during the run.

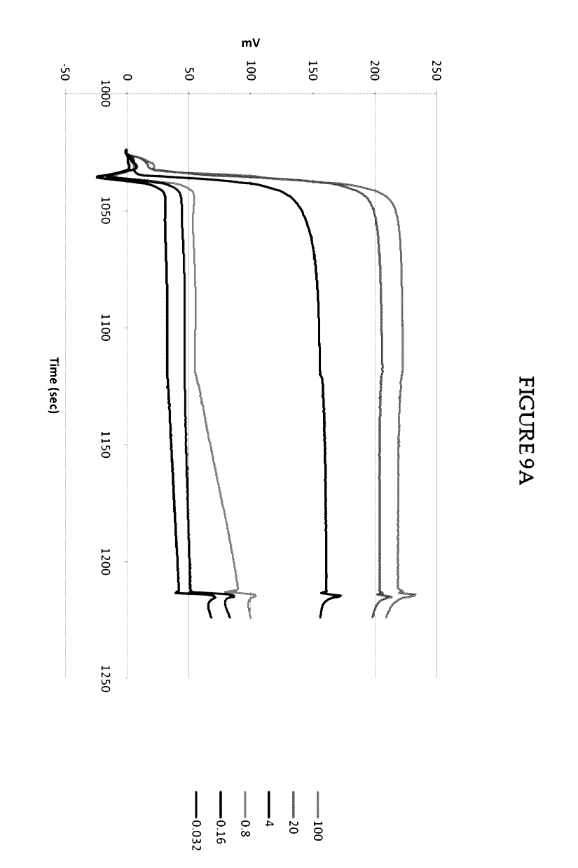

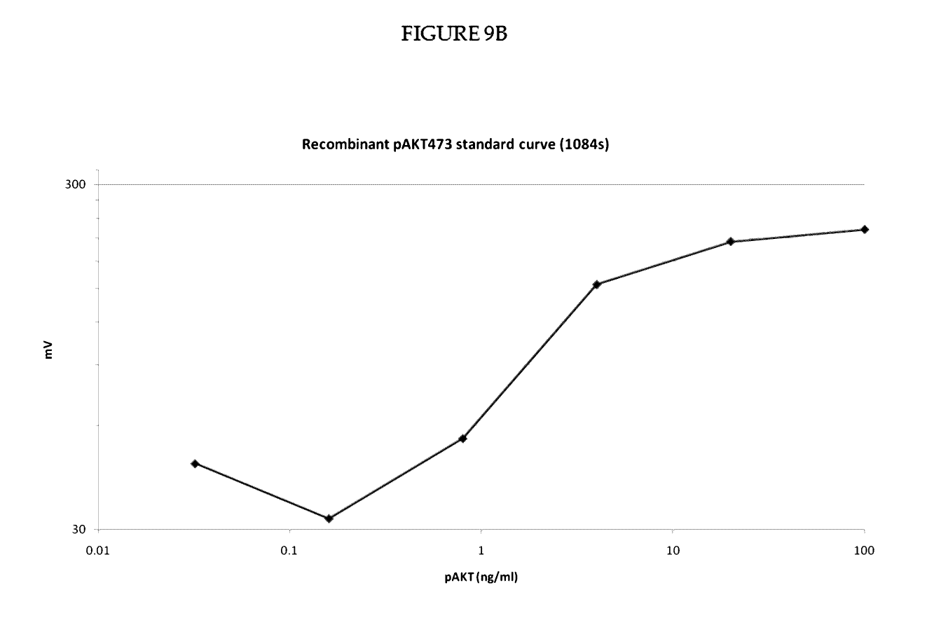

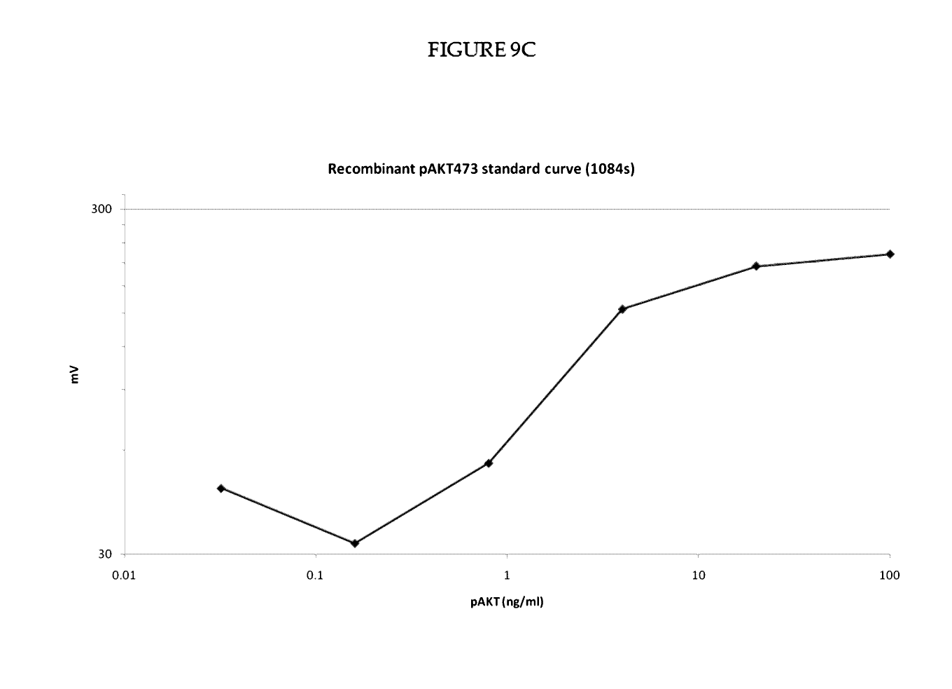

The sample/reaction buffer mix was then run on a microfluidic cartridge as described in Example 4. The results are shown in FIGS. 9A-9C. Each data point shown is the average of 3 flow cells from a single cartridge. The data was transformed by taking the point at which substrate injection begins as zero. Data was collected from the point at which substrate flow through begins up until the end of substrate incubation phase (180 s after substrate injection).

Using the data taken from 180 seconds after injection of substrate, the detection limit of the chip was about 1 ng/ml pAKT.

### Example 7

### Reagent Order of Addition Permutations

Capture antibody (anti-pERK-peptide conjugate or anti-pERK-biotin conjugate), detection antibody (anti-total ERK-HRP conjugate), capture/detection antibody mixture, and varying concentrations of cell lysate containing pERK were added to (A) anti-peptide conjugate antibody coated plates or (B) streptavidin coated microplates, in 8 different permutations (refer to Table 1 & 2). Individual assay components were added 1 min apart to the plates, and incubated for 2 h. Plates were washed, incubated with HRP substrate, before detection of the fluorescent product.

TABLE 1
Reagent volumes for order of addition assessment

| Assay Component | Volume/Well |
|---|---|
| Capture/Detection Antibody Mix | 50 ul |
| Lysate | 50 ul |
| Capture Antibody | 25 ul |
| Detection Antibody | 25 ul |

TABLE 2
Reagent order of addition permutations

| Trial # | 1^{st} Addition | 2^{nd} Addition | 3^{rd} Addition |
|---|---|---|---|
| 1 | Capture/Detection Ab | Lysate | n/a |
| | Mix | | |
| 2 | Lysate | Capture/Detection Ab | n/a |
| | | Mix | |
| 3 | Lysatc | Capture Ab | Detection Ab |
| 4 | Lysate | Detection Ab | Capture Ab |
| 5 | Detection Ab | Capture Ab | Lysate |
| 6 | Detection Ab | Lysate | Capture Ab |
| 7 | Capture Ab | Lysate | Detection Ab |
| 8 | Capture Ab | Detection Ab | Lysate |

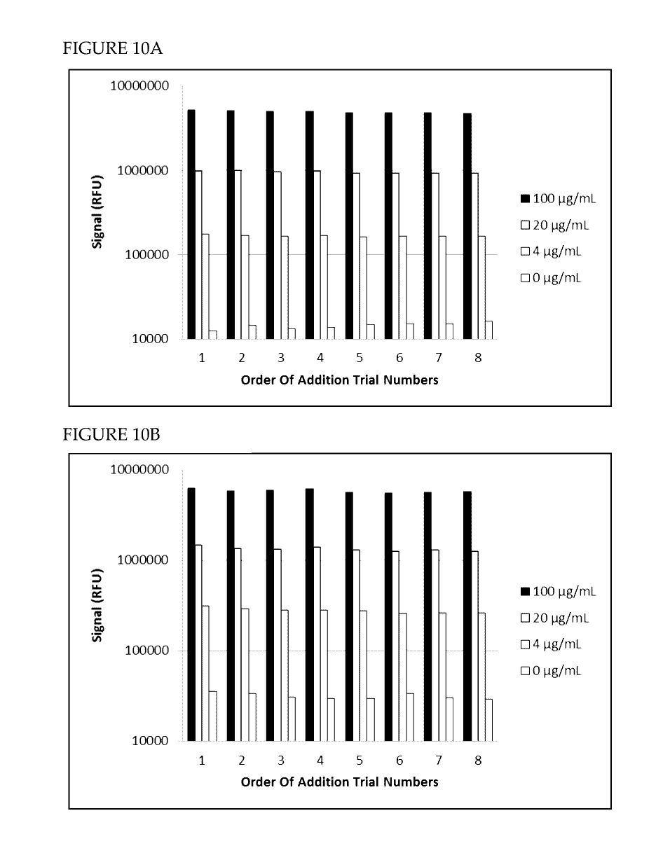

The effect of reagent order of addition on pERK detection in the single-incubation ELISA using different capture systems is shown in FIG. 10. Across the 8 different permutations, and at several analyte concentrations, little signal difference were observed. This result demonstrates that equivalent results can be obtained in a single-incubation ELISA assay, irrespective of the order of addition of the individual components.

### Example 8

### Recombinant Protein Standard Curves in Different Biological Milieu Using the Peptide Conjugate Capture System

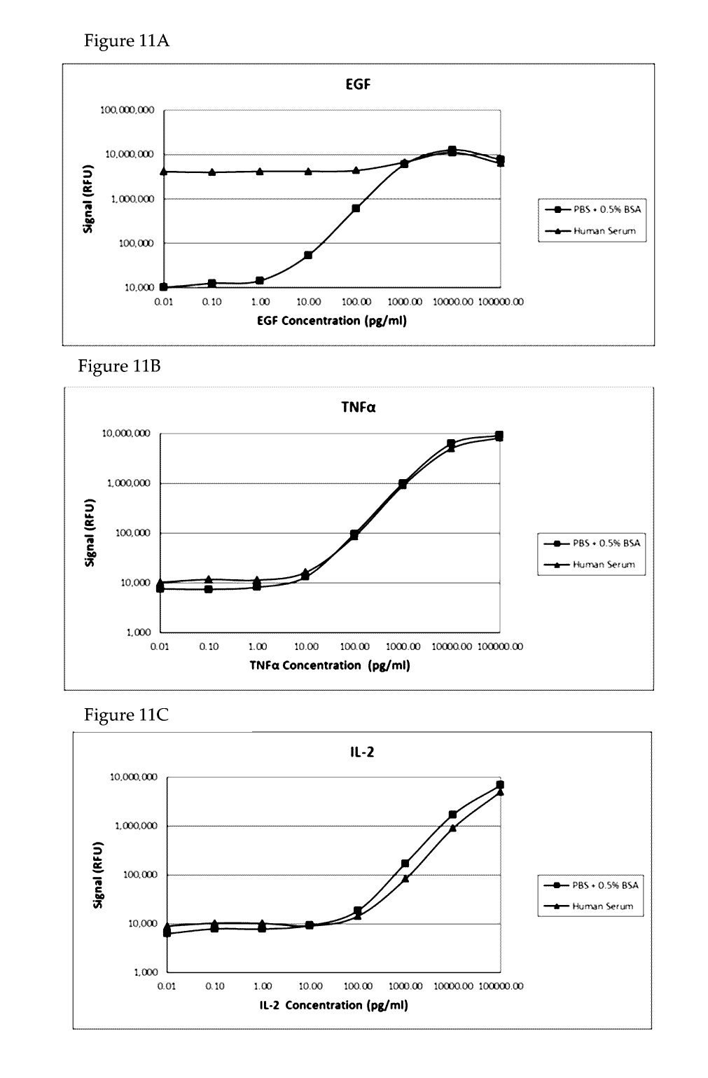

A demonstration of the use of the single-incubation ELISA assay format for the detection of three recombinant human proteins diluted in human serum is provided in FIG. 11. EGF, IL-2 and TNFα were measured in PBS/0.5% BSA and human serum. Detection limits of ≦10 pg/mL were ascertained for each assay in PBS/0.5% BSA, and similar sensitivity for both IL-2 and TNFα were observed for analyte diluted in human serum. The detection limit for EGF in human serum could not be detected due to the presence of a high level of endogenous EGF, which was confirmed using a standard commercial EGF ELISA kit (R&D Systems, data not shown). FIG. 11 shows the mean and standard deviations for the duplicate data points for each target analyzed.

This data illustrates that the single-incubation ELISA assay format was robust to measuring analytes in different biological milieu. The assay clearly demonstrates efficacy for several different targets in serum, whereby the assay components are incubated concurrently. The high signal for EGF in human serum is due to the presence of endogenous EGF protein(s) in this medium.

### Example 9

### Recombinant Protein Standard Curves Using the Peptide Conjugate Capture System in a 10 Min Single-Incubation ELISA

Nunc 96 well Maxisorp™ plates were passively coated with an anti peptide tag antibody overnight at 4° C. Plates were washed 3× with PBS-T and blocked with 200 μL/well of a 1% BSA solution in PBS-T (0.05%). Blocking solution was aspirated prior to assay. Analyte (eg 50 μL of recombinant protein) were added to the wells followed by the addition of an antibody antibody mixture (50 μL) containing pre-optimised concentrations of peptide tag conjugated anti-analyte capture antibody and HRP-conjugated anti-analyte detection antibody. Plates were incubated for 10 min before washing 3× with PBS-T. Fluorescent HRP substrate (100 μL) was added to the wells and incubated for 5 mins before measurement of fluorescent product.

Recombinant human proteins EGF, IL-2 and TNFα were prepared in PBS/0.5% BSA at concentrations ranging from 100 ng/mL down to 1 pg/mL and 50 μL/well added to an anti peptide tag antibody ELISA plate. Capture/detection antibody mix for EGF (A), IL-2 (B) and TNFα (C) were added to the appropriate ELISA plate wells and incubated for 10 min. Plates were washed before incubation with HRP substrate for 5 min and detection of the fluorescent product.

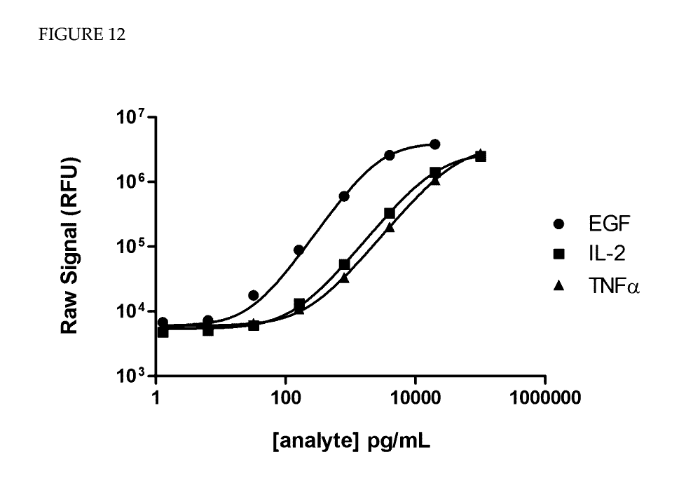

FIG. 12 shows the detection of three recombinant human proteins using a 10 min single-incubation ELISA assay format on an anti peptide tag antibody coated ELISA plate. EGF, IL-2 and TNFα standard curves were measured successfully in PBS/0.5% BSA with detection limits of ≦32 pg/mL ascertained for each assay. This data illustrated that the simplified peptide conjugate capture/single-incubation ELISA assay format was amenable to measuring multiple analytes on the same plate in as little as 10 minutes. As can be seen, in the 10 minute single-incubation ELISA the assay was still able to efficiently detect the three analytes, even at a concentration of the analytes less than 100 pg/ml.

### Example 10

### Intra-Plate Variation

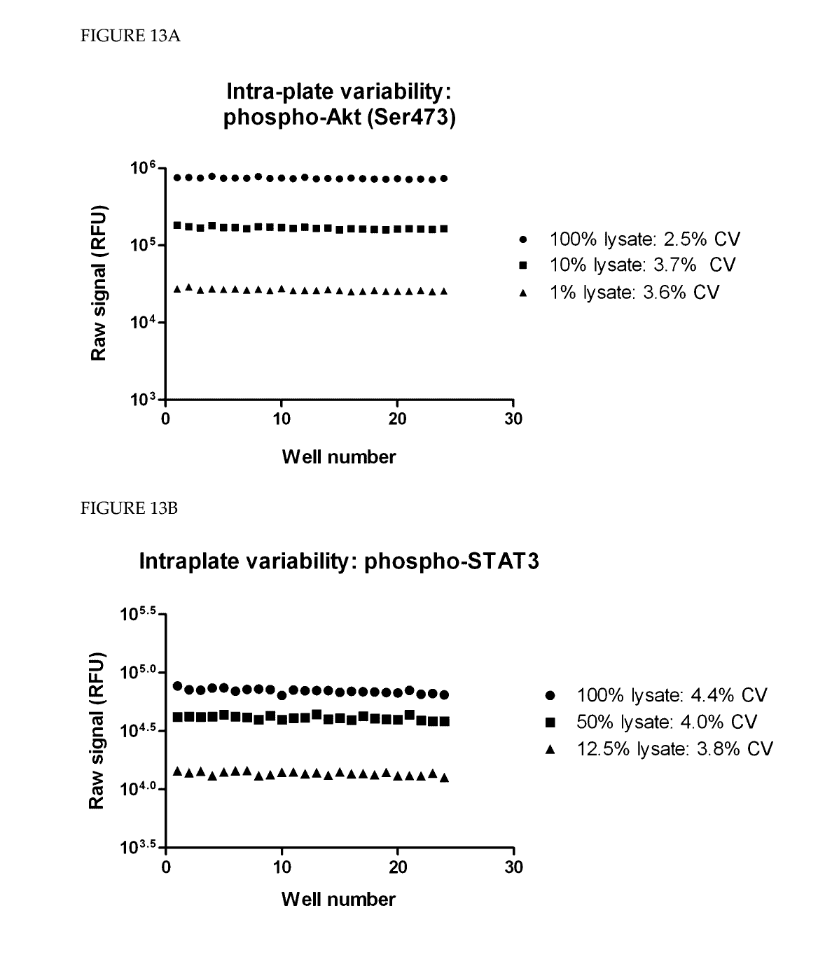

FIG. 13 shows intra-plate variation observed for 2 separate single-incubation ELISAs for either phospho-AKT (pSer473) or phospho-STAT3. For each target, cellular lysate was diluted to 3 different concentrations using 1× Lysis buffer as indicated, and added to 24 replicate wells of a 96-well streptavidin-coated microplate. To initiate the assay reaction, for either target, a mixture of the biotin-conjugated capture antibody, and the HRP-conjugated detection antibody were added to the lysates, and incubated for 1 hour. The wells were subjected to a standard wash cycle for each assay. After the wash cycle, QuantaRed™ HRP substrate was added to the wells, and each plate was incubated for 10 min in the dark. The fluorescent signal in the wells was measured at 550ex/600em nm. FIGS. 13A and 13B show the data points at each lysate concentration analyzed, for phospho-AKT and phospho-STAT3, respectively. The coefficient of variation (CV %) for each analyte concentration was calculated by dividing the standard deviation observed over the 24 wells at each concentration, by the mean of the 24 wells at the same concentration, and transforming this fraction to a percentage value. Typically, a value of less than 10% is desired for many assays, for example, in certain high quality assays, and the data presented here demonstrates suitable low intra-plate variability characteristics.

### Example 11

### Detection of TNFα

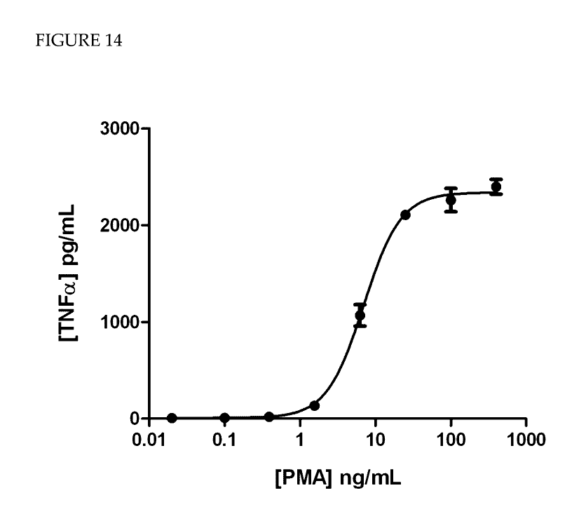

FIG. 14 shows detection of TNFα in tissue culture supernates. THP-1 cells were seeded into 96-well tissue culture microplates in RMPI cell culture medium containing 10% (v/v) foetal bovine serum and various other standard cell culture additives. The cells were then treated with a various concentrations of PMA diluted in the same medium, and incubated overnight in a humidified 37° C. incubator. The following day 50 μL of medium was aspirated from the cell culture wells, and added to the wells of a peptide-coated 96-well assay plate. The assay reaction was initiated by the addition of 50 μL of an antibody mixture containing the capture antibody-peptide conjugate, and the detection antibody-HRP conjugate, and incubated for 1 hour. The wells were subjected to a standard wash cycle for each assay. After the wash cycle, fluorescent HRP substrate was added to the wells, and each plate was incubated for 10 min in the dark. The fluorescent signal in the wells was measured at 540ex/590em nm, and quantitated using a standard curve generated against the same target. FIG. 14 shows the mean and standard deviations for the duplicate data points for each target analyzed. In this Figure, the assay demonstrates efficient detection of specific target analyte in tissue culture supernates using the certain embodiments, whereby the assay components are incubated concurrently.

### Example 12

Detection of Phospho-AKT (pSer473) or Phospho-ERK in a 25 Min Total Assay Time

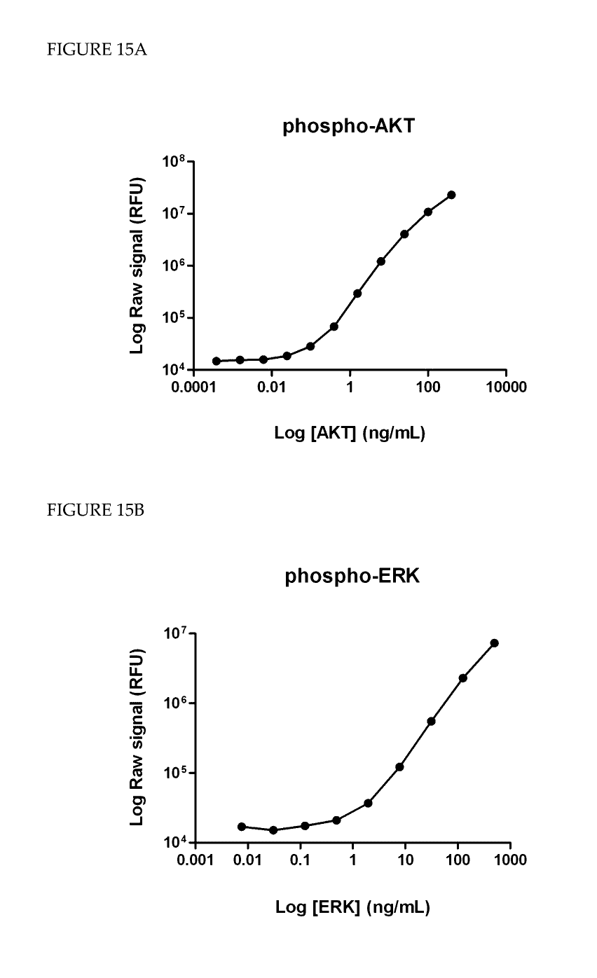

FIG. 15 shows detection of either phospho-AKT (pSer473) or phospho-ERK in a 25 min total assay time. For each target, recombinant active (A) phospho-AKT or (B) phospho-ERK was diluted as indicated, to various concentrations using 1× Lysis buffer containing 0.1% BSA and added to 4 replicate wells of a 96-well streptavidin-coated microplate. To initiate the assay reaction, for either target, a mixture of the biotin-conjugated capture antibody, and the HRP-conjugated detection antibody were added to the lysates, and incubated for 1 hour. The wells were subjected to a standard wash cycle for each assay. After the wash cycle, QuantaRed™ HRP substrate was added to the wells, and each plate was incubated for 10 min in the dark. The fluorescent signal in the wells was measured at 550ex/600em nm. FIGS. 15A and 15B show the data points at each analyte concentration analyzed, for phospho-AKT and phospho-ERK, respectively. Both assays demonstrated sensitivity to less than 1 ng/mL.

### Example 13

### Detection of IL-2 in Using a ERK Peptide-Anti Peptide Capture Pair

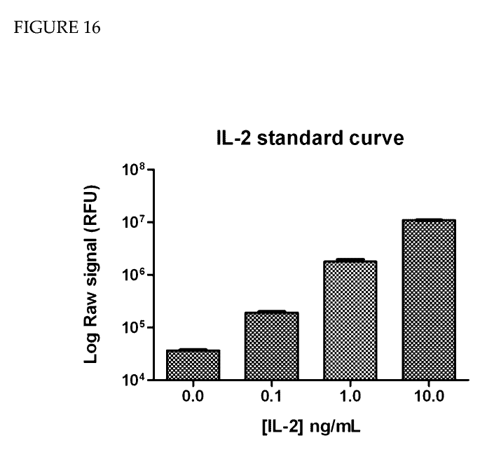

FIG. 16 shows detection of IL-2 in using a ERK peptide-anti peptide capture pair. Recombinant interleukin 2 (IL-2) was diluted as indicated, to various concentrations using 1×PBS containing 0.1% BSA and added to duplicate wells of a 96-well anti-ERK-peptide antibody-coated microplate. To initiate the assay reaction, for either target, a mixture of the ERK peptide capture antibody, and the HRP-conjugated detection antibody were added to the lysates, and incubated for 1 hour. The wells were subjected to a standard wash cycle for each assay. After the wash cycle, fluorescent HRP substrate was added to the wells, and each plate was incubated for 10 min in the dark. The fluorescent signal in the wells was measured at 540ex/590em nm. FIG. 16 shows the data points at each analyte concentration analyzed, demonstrating sensitivity to 100 pg/mL or less.

### Example 14

### General Discussion

The single-incubation ELISA uses an immuno-sandwich format, but with at least one difference. For the single-incubation ELISA assay, both the analyte and the assay reagents are added to the assay microplate at the same time, in solution. After a short incubation period, unbound assay reagents and analytes are washed away, and immuno-complexes containing both antibodies are detected. The single-incubation ELISA allows the user a higher degree of assay flexibility. In contrast to other ELISA formats, in particular sets of examples no target-specific antibodies are present on the assay microplate itself, so assays for several different targets can be performed in different wells on the same microplate. For example, a cellular lysate can be analyzed on the same assay microplate in parallel for p38-MAPK phosphorylation, ERK phosphorylation, AKT phosphorylation and JNK phosphorylation, giving fast, accurate and quantifiable information on key cell signalling events. However, if desired target antibodies may be immobilized on the plate.

The single-incubation ELISA provides the high quality results desired from a sandwich immunoassay, and the assay allows for the use of self-contained kits to conduct the assay.

For example, a kit may contain one or more of the following components:
Capture Antibody Reagent
Detection Antibody Reagent
Lysis Buffer (for example supplied at 5× concentration) containing a mixture of detergents for cellular lysis, and phosphatase inhibitors.
Enhancer Solution—containing factors for enhancing assay performance, such as anti-HAMA components, and target-specific additives to increase assay performance.
ADHP Dilution Buffer—containing cofactors necessary for the HRP-mediated conversion of ADHP to resorufin.
ADHP (for example supplied at 100× concentration)
Wash Buffer (for example supplied at 10× concentration)
Stop Solution—for stopping HRP activity when necessary
Assay Control Lysate
Assay microplate
Assay diluent—for the dilution of concentrated samples

### Example 15

### General Assay Protocols

(i) Protocol for Use with Samples Such as Cellular Lysates and Tissue Culture Supernates

### Assay Protocol

1. Add 50 ul/well of sample to the assay microplate. 50 ul/well assay controls may be added to separate wells if desired.

2. Add 50 ul/well of antibody mix to the wells. Generally a concentration of antibodies in the mix of 50-500 ng/mL is suitable. Cover the microplate and incubate at room temp on a microplate shaker (−300 rpm).

3. Wash wells with 200 ul/well wash buffer (repeat 3 times). After final wash, remove any remaining wash solution from wells. A suitable wash buffer is PBS containing Tween 20.

4. Immediately prior to use, prepare substrate mix. A suitable substrate mix is TMB, ADHP, OPD, or other suitable HRP substrates, diluted with co-factors suitable for mediating their conversion to measurable by-products. Add 100 ul/well of substrate mix. Cover microplate with foil, and incubate for 10 minutes at room temp on a microplate shaker (−300 rpm).

5. Add 10 ul/well stop solution, and mix briefly (5-10 sec) on a microplate shaker. A suitable stop solution is a dilute acid such as HCl, or a strong detergent such as SDS.

6. Read fluorescence signal with a compatible filter set.

(ii) Protocol for Serum Samples, or Other Samples that May Carry Sample-Specific Interferences

### Assay Protocol

1. Add 25 ul/well Enhancer mix. Enhancer mix containing general components for the neutralization of HAMAs, as well other components for the neutralization of target-specific binding proteins carried in serum.

2. Add 50 ul/well of sample to the assay microplate. 50 ul/well assay controls may be added to separate wells if desired.

3. Add 25 ul/well of antibody mix to the wells. Cover the micro plate and incubate for 1 hour at room temp on a microplate shaker (−300 rpm).

3. Wash wells with 200 ul/well wash buffer (repeat 3 times). After final wash, remove any remaining wash solution from wells.

4 Prepare substrate prior to use and add 100 ul/well. Cover microplate with foil, and incubate for 10 minutes at room temp on a microplate shaker (−300 rpm).

5. Add 10 ul/well stop solution, and mix briefly (5-10 sec) on a microplate shaker.

6. Read fluorescence signal with a compatible filter set.

### Example 16

### Detection of Various Concentrations of IL-2 Using a Peptide Tag Anti Peptide Tag Antibody Capture System

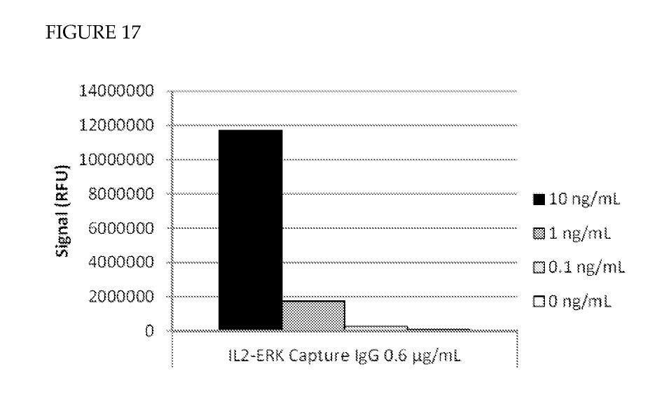

FIG. 17 shows detection of various concentrations of IL-2 using a peptide tag anti peptide tag antibody capture system.

Antibodies were generated in mice as monoclonal antibodies to a 23 amino acid peptide, KRITVEEALAHPYLEQYYDPTDE (SEQ ID NO.2), a sequence derived from the carboxy terminus of the human ERK proteins (ERK C-term peptide). Purified antibodies (TGR, 12D4) to this peptide were passively coated onto a maxisorb Nunc immunoassay plate, and the plate then blocked against further non-specific protein attachment. The ERK C-term peptide was also used to conjugate to antibodies to the human IL-2 protein (R&D Systems), so that the peptide would act to anchor this antibody to the plate surface. A second IL-2 antibody (R&D Systems) was conjugated to horse radish peroxidase (HRP) to be used as the reporter antibody. Recombinant human IL-2 was mixed with PBS/BSA (0.1%) at various concentrations shown, and to these solutions were added the IL-2 antibodies. After an hour incubation, the wells were washed with a wash buffer, and fluorescent HRP substrate added for 10 min, followed by reading of the plate at 540/590 nm ex/em wavelengths in a plate reader.

It can be seen that the assay system measured the concentrations of IL-2 present in each sample and that the variation between samples was low as indicated by the small error bars.

### Example 17

### Detection of Various Concentrations of EGF, IL-2 & TNFα Using a Peptide Tag Anti Peptide Tag Antibody Capture System

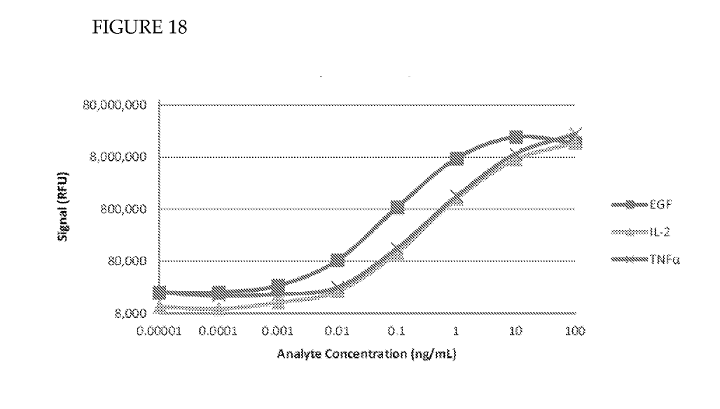

FIG. 18 shows detection of various concentrations of EGF, IL-2 & TNFα using a peptide tag anti peptide tag antibody capture system.

Antibodies specific to the peptide DYKDDDDK (SEQ ID NO.1; Sigma, catalog number F1804) were passively coated onto a maxisorb Nunc immunoassay plate at 5 μg/mL overnight in PBS, and the plate then blocked against further non-specific protein attachment. The peptide DYKDDDDK (SEQ ID NO.1) was also used to conjugate to IgG antibodies to the human IL-2 protein (R&D Systems), human EGF or human TNFα so that the peptide would act to anchor this antibody to the plate surface. A second detectable antibody to each analyte (R&D Systems) was also conjugated to horse radish peroxidase (HRP) to be used as the reporter antibody. EGF, IL-2 & TNFα peptide (C-terminal acid) capture IgG's & and their respective HRP detection were IgG's prepared in reaction buffer. Pure analytes as standards were diluted in PBS/BSA (0.5%) at various concentrations shown. Analyte (50 μL/well) was added to the coated plate and then added 50 μL/well of corresponding antibody mix (Capture 200 ng/mL; detection 50 ng/mL). After an hour incubation with shaking, the wells were washed three time with a wash buffer, and fluorescent HRP substrate (ADHP) added for 10 min, followed by reading of the plate at 540/590 nm ex/em wavelengths in a plate reader. The data shows the sensitive detection of each of EGF, IL-2 and TNFα in separate wells of a microtitre plate using a single-wash, peptide tag antibody capture system.

### Example 18

### Detection of Various Concentrations of Analyte Using a Peptide Tag Anti Peptide Tag Antibody Capture System

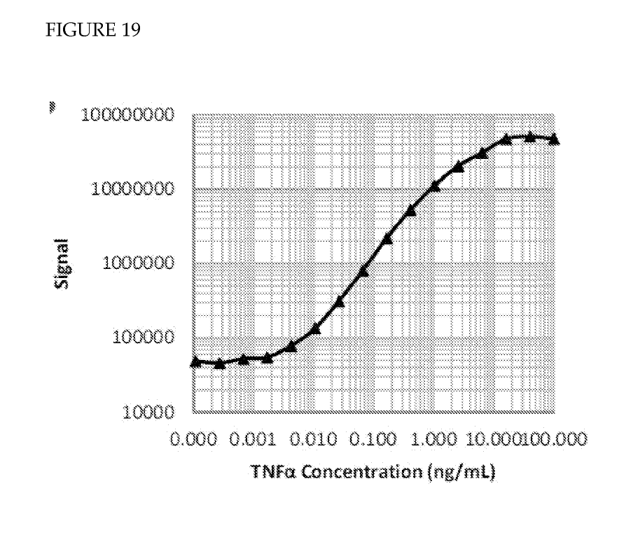

FIG. 19 shows the signal obtained for various concentrations of analyte using a peptide tag anti peptide tag antibody capture system.

Antibodies were generated in mice as monoclonal antibodies to the peptide DYKDDDDK (SEQ ID NO.1). Purified antibodies to this peptide were coated onto a maxisorb Nunc immunoassay plate at 10 ug/ml, and the plate then blocked against further non-specific protein attachment. The peptide DYKDDDDK (SEQ ID NO.1) was also used to conjugate to antibodies to the human TNFα protein (R&D Systems), so that the peptide would act to anchor this antibody to the plate surface. A second TNFα antibody (R&D Systems) was conjugated to horse radish peroxidase (HRP) to be used as the reporter antibody. TNFα was mixed with PBS/BSA (0.5%) at various concentrations shown, and to these solutions were added the IL-2 antibodies (Capture 200 ng/mL; detection 50 ng/mL). After an hour incubation with shaking, the wells were washed with a wash buffer, and fluorescent HRP substrate added for 10 min, followed by reading of the plate at 540/590 nm ex/em wavelengths in a plate reader. The data shows that the use of a peptide tag antibody capture system, whereby in this case the peptide tag was DYKDDDDK (SEQ ID NO.1), and the system was a single-wash ELISA format, enabled the sensitive measurement of TNFα with a total assay time of approximately 1 hour.

### Example 19

### Comparison of a Biotin-Streptavidin Capture System to a Peptide Tag-Anti-Peptide Antibody Capture System in Various Biological Milieu

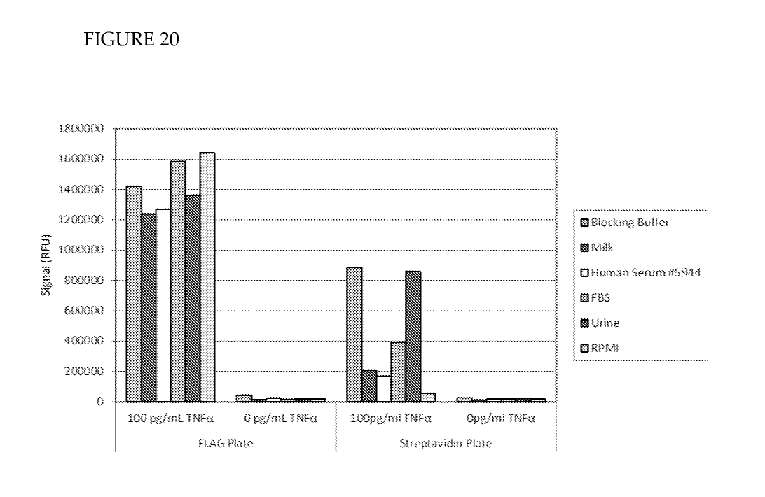

FIG. 20 shows a comparison of a biotin-streptavidin capture system to a peptide tag-anti-peptide antibody capture system in various biological milieu.

Antibodies were generated in mice as monoclonal antibodies to the peptide DYKDDDDK (SEQ ID. NO.1). Purified antibodies to this peptide were coated onto a maxisorb Nunc immunoassay plate at 10 ug/ml overnight in carbonate buffer, and the plate washed and then blocked against further non-specific protein attachment. Separately, a commercial streptavidin-coated plate (Nunc Immobiliser) was used for biotin-conjugated antibodies assays. The peptide DYKDDDDK (SEQ ID NO.1) was used to conjugate to antibodies to the human TNFα protein (R&D Systems), so that the peptide would act to anchor this antibody to the plate surface to which had been coated antibodies to this peptide. Separately, antibodies to the human TNFα protein (R&D Systems), were also linked with biotin, so that this would act to anchor this antibody to the plate surface to which had been coated streptavidin. A second species of TNFα antibody (R&D Systems) was conjugated to horse radish peroxidase (HRP) to be used as the reporter antibody. TNFα was mixed with various media (blocking buffer, milk, human serum, FBS, urine or RPMI) at 100 pg/mL or not added at all, and to these solutions were added either to the TNFα antibodies linked with biotin (Capture 750 ng/mL; detection 50 ng/mL) or peptide DYKDDDDK (SEQ ID NO.1) (Capture 300 ng/mL; detection 50 ng/mL), and the HRP-linked TNFα antibodies. After an hour incubation, the wells were washed with a wash buffer, and fluorescent HRP substrate ADHP added for 10 min, followed by reading of the plate at 540/590 nm ex/em wavelengths in a plate reader. It can be seen from the data that the peptide tag-anti-peptide antibody capture systems was superior to the biotin-streptavidin system in detecting analytes, particularly when analytes were present in particular media. Of special note are the inhibitory effects on the assay of TNFα present in milk, serum, FBS and RPMI when using the biotin-streptavidin system, reflecting the presence of biotin in these samples that interferes with this capture system.

### Example 20

### Streptavidin Biotin Capture Systems Utilizing an Antibody Capture Agent and an Antibody Detectable Agent is not Affected by Increasing Concentrations of Irrelevant Antibodies

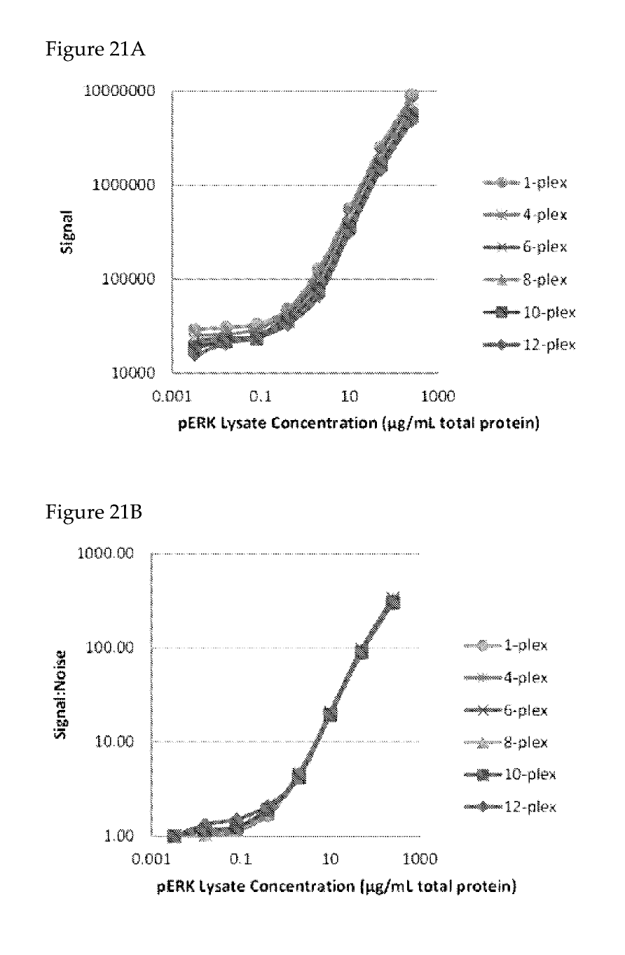

FIG. 21A shows that a streptavidin biotin capture system utilizing an antibody capture agent and an antibody detectable agent is not affected by increasing concentrations of irrelevant antibodies.

Nunc Immobiliser plates, coated with streptavidin, were used in an assay to determine capacity of p-ERK antibody binding and p-ERK analyte measurement. Antibodies to the phosphorylation site of the ERK protein (TGR, Thr202/Tyr204) were linked with biotin, so that this would act to anchor this antibody to the plate surface to which has been coated streptavidin. Separately, a second ERK antibody (Santa Cruz) was linked to horse radish peroxidase (HRP) to act as a reporter antibody. Samples containing cellular lysates in which the p-ERK protein was present at various concentrations were then mixed with the ERK antibodies either in the absence (1-plex) or presence (4-12-plex) of increasing numbers of pairs of irrelevant antibodies at the same concentration as the ERK antibodies, such that one of the pair of the irrelevant antibodies was also biotinylated in the same way and extent as the ERK antibody. After 1 hour, the wells were washed with a wash buffer, and fluorescent HRP substrate ADHP added for 10 min, followed by reading of the plate at 540/590 nm ex/em wavelengths in a plate reader. Results are presented as absolute fluorescence signal.

FIG. 21B shows the data from FIG. 21A has been normalised in terms of signal:noise, where noise is the signal of the immunocomplex obtained for each condition compared to the signal obtained in the absence of analyte.

It can be seen from these graphs that the single-wash assay system with both antibodies being present with the analyte, in this case using the biotin-streptavidin pair, can use low concentrations of Capture antibodies, allowing the presence of up to 12 pairs of unrelated tagged antibodies to be present without there being any assay interference.

### Example 21

### Anti Peptide Tag Antibody-Peptide Capture Systems Utilizing an Antibody Capture Agent and an Antibody Detectable Agent is not Affected by Increasing Concentrations of Irrelevant Antibodies

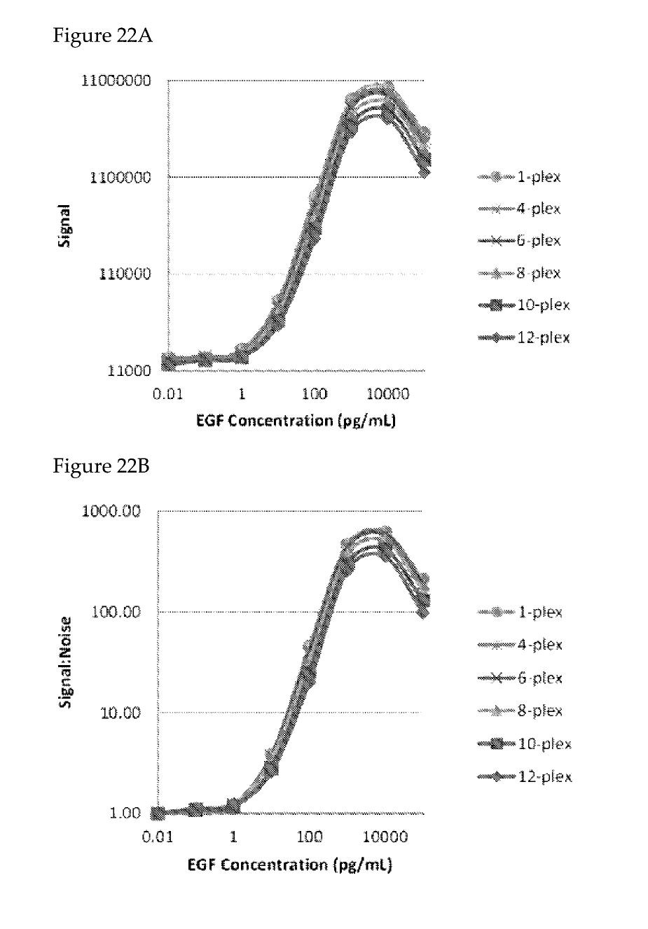

FIG. 22A shows that anti peptide tag antibody-peptide capture system utilizing an antibody capture agent and an antibody detectable agent is not affected by increasing concentrations of irrelevant antibodies

Antibodies were generated in mice as monoclonal antibodies to the peptide DYKDDDDK (SEQ ID NO.1). Purified antibodies to this peptide were coated onto a maxisorb Nunc immunoassay plate, and the plate then blocked against further non-specific protein attachment. Antibodies to the human EGF protein (R&D Systems) were linked with the peptide DYKDDDDK (SEQ ID NO.1), so that this would act to anchor this antibody to the plate surface to which has been coated streptavidin. Separately, a second EGF antibody (R&D Systems) was linked to horse radish peroxidase (HRP) to act as a reporter antibody. Samples containing EGF at various concentrations were then mixed with the EGF antibodies either in the absence (1-plex) or presence (4-12-plex) of increasing numbers of pairs of irrelevant antibodies at the same concentration as the EGF antibodies, such that one of the pair of the irrelevant antibodies was also linked with the peptide DYKDDDDK in the same way and extent as the EGF antibody. After 1 hour, the wells were washed with a wash buffer, and fluorescent HRP substrate ADHP added for 10 min, followed by reading of the plate at 540/590 nm ex/em wavelengths in a plate reader. Results are presented as absolute fluorescence signal.

FIG. 22B shows the data from FIG. 22A has been normalised in terms of signal:noise, where noise is the signal of the immunocomplex obtained for each condition compared to the signal obtained in the absence of analyte.

It can be seen from these graphs that the single-wash assay system with both antibodies being present with the analyte, in this case using the peptide-anti-peptide antibody pair, can use low concentrations of Capture antibodies, allowing the presence of up to 12 pairs of unrelated tagged antibodies to be present without there being any assay interference.

### Example 22

### Multiplex Detection of Analytes

To demonstrate the ability to perform multiplex detection of two analytes, experiments were performed to detect the presence of IL2 and EGF at various concentrations by ELISA and to demonstrate that similar data were obtained when these analytes were either measured singly in each assay well or dually with two different detection tags.

EGF and IL2 were chosen as a two representative analytes for detection. Each was assayed using dual antibodies for each target, one antibody of each pair being labelled with a different lanthanide (either Europium or Samarium). The other antibody of each pair was tagged with a peptide conjugate (FLAG peptide) to facilitate capture and immobilisation on the plate coated with an anti-peptide antibody (anti-FLAG peptide antibody).

Antibodies were labelled with lanthanide with the following PerkinElmer labelling kits as per the protocols provides:

Eu-labelling kit, cat number 1244-302—used to label the EGF antibody.

Sm-labelling kit, cat number 1244-303—used to label the IL2 antibody.

Antibodies were incubated with the respective lanthanide solutions for 16 hours at room temperature, and conjugates were then desalted using a PD 10 column.

Standard curves for each conjugate were constructed using log 00 dilutions of the conjugates, maximal concentrations for Eu and Sm being 10 nM and 100 nM, respectively. The time-resolved fluorescence readings for these solutions were assessed in microtitre plates in a Victor II plate reader (PerkinElmer). Excitation and emission wavelengths for these lanthanides were: Europium: Excitation 340 nm/Emission 615 nm; Samarium: 340 nm/Emission 642 nm.

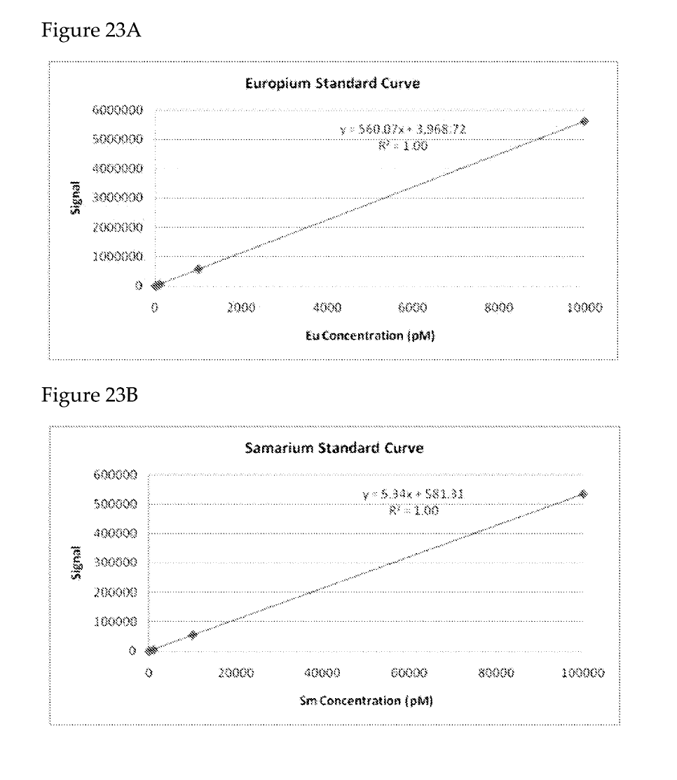

The data is shown in FIG. 23.

It can be seen that the signal from Samarium is 1-2 log 10 less at each concentration than Europium, which is consistent with the known fluorescence properties of these two molecular tags.

To perform the mono- and duo-plexing experiment for EGF and IL2, each pair of antibodies was either incubated alone with single analyte to detect (EGF or IL2), or with both antibody pairs together in the same well with both analytes.

As described, the capture antibodies for each analyte were conjugated with the FLAG peptide tag. The base of each assay well was coated with an antibody to FLAG, allowing the capture antibodies to be specifically immobilised out of solution onto the base of the plate. The detection antibody did not have the tag, and its binding and immobilisation to the base of the plate was, therefore, dependent on binding the analyte of interest.

The capacity of binding of the tagged antibodies to the base of the plate, therefore, was dependent on the binding capacity of the FLAG antibody immobilised on the base of the assay well. This binding capacity had been predetermined to be greater than the amount of tagged antibodies being presented to the well in the assays.

Antibodies were present at concentrations of 50 ng/mL and 25 ng/mL, for capture and detection antibodies, respectively.

In addition to measuring EGF and IL2 using antibodies tagged with the lanthanides, for comparison the same analytes were measured in separate wells of the assay plate with detection antibodies that were conjugated with HRP. In this case, the assay readout was from the enzymatic conversion of ADHP (10-Acetyl-3,7-dihydroxyphenoxazine) to resorufin. The latter was detected using standard fluorescence settings for this molecule.

The protocol of the experiment was as follows:

1. Prepare analyte(s) for measurement in PBS/BSA solution

2. Add analyte to assay plate (pre-coated with FLAG antibody) at various concentrations in different wells

3. Add both antibodies to the analyte(s) of interest to sample in wells. For single analyte detection, either EGF or IL2 antibody pair were added. For duoplex detection of both EGF and IL2 in the same well, both antibody pairs were added.

4. Incubate plate for 1 hour

5. Wash plate 3 times with PBS

6. For those samples where the detection antibody was HRP-tagged, ADHP was added and incubated for 10 min.

7. Read plate for time-resolved fluorescence at Eu and Sm wavelengths, or fluorescence (ADHP samples), depending on the detection antibody conjugate present.

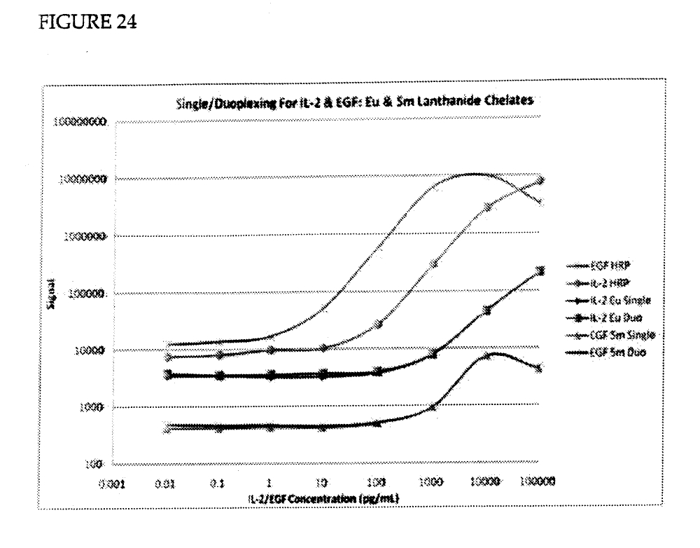

The data is shown in FIG. 24.

Referring to FIG. 24, the light blue line shows detection of EGF using antibodies conjugated with HRP. The orange line shows detection of IL2 using antibodies conjugated with HRP. The dark blue line shows detection of IL2 with europium tagged antibodies in a monoplex situation using europium wavelength settings on the plate reader. The red line shows detection of IL2 with europium-tagged antibodies using europium settings on the plate reader in the presence of the samarium-tagged antibodies specific for EGF. The green line shows monoplex detection of EGF with antibodies tagged with samarium, using plate reader wavelength settings specific for samarium detection. The purple line shows EGF detection with antibodies for EGF tagged with samarium using plate reader wavelength settings specific for samarium detection, in the presence of antibodies tagged with europium that are specific for IL2. For those samples measuring EGF and IL2 in the duoplex format, the readings for both EGF and IL2 have been made sequentially from the same wells containing both pairs of antibodies for each analyte.

The results demonstrate the detection of two analytes in a multiplex setting, with no apparent interference between antibodies and or lanthanide signalling.

### Example 23

### Duoplex Detection Utilising Enzyme Tagged Detection Antibodies

For detection of two analytes in a single well, utilising an alternative detection system to that with lanthanides, secondary antibodies coupled to enzymes may be utilised. Similar to the method described in Example 22, primary antibodies may be tagged with the FLAG peptide to enable localisation to the base of the assay well, which has been pre-adsorbed with the anti-FLAG antibody. Using the example of the detection of EGF and IL-2, antibodies to each of these analytes may be tagged with FLAG peptide, as described above. The secondary antibodies to each analyte may be labelled with horse radish peroxidase (HRP) and alkaline phosphatase (AP), respectively.

To each assay well of a 96 well plate sample may be added to test for the presence of either EGF or IL-2. To each well is also be added both pairs of capture and detection antibody. After incubation for a period of 1 hour, the wells are washed 3 times with PBS, and then to the wells is added substrates for both HRP and AP. These substrates may be utilised as the emission characteristics of their products have a fluorescence signal that does not significantly overlapping with each other. Filter sets on the plate reader used to measure the fluorescence are chosen to provide wavelength cutoffs that allow clear measurement of each fluorescence peak. After a period of 15 minutes, the reactions in each well are stopped by the addition of a stop solution, and the fluorescence in each well determined.

### Example 24

### Trioplex Detection Utilising Enzyme Tagged Detection Antibodies

For detection of three analytes in a single well, utilising an alternative detection system to that with lanthanides, secondary antibodies coupled to enzymes are utilised. Similar to the method described in Example 23, primary antibodies are tagged with the FLAG peptide to enable localisation to the base of the assay well, which had been pre-adsorbed with the anti-FLAG antibody. Using the example of the detection of EGF, IL-2 and TNFα, antibodies to each of these analytes may be tagged with FLAG peptide, as described above. The secondary antibodies to each analyte are labelled with horse radish peroxidase (HRP), alkaline phosphatase (AP), and beta-galactosidase (bGAL), respectively.

To each assay well of a 96 well plate, sample may be added to test for the presence of EGF, IL-2 or TNFα. To each well is also added pairs of capture and detection antibody for each analyte. After incubation for a period of 1 hour, the wells were washed 3 times with PBS, and then to the wells is added substrates for HRP, AP and bGAL. These substrates are selected by the emission characteristics of their products having a fluorescence signal that does not significantly overlap with each other. Filter sets on the plate reader used to measure the fluorescence are chosen to provide wavelength cutoffs that allowed clear measurement of each fluorescence peak. After a period of 15 minutes, the reactions in each well are stopped by the addition of a stop solution, and the fluorescence in each well determined.

### Example 25

### Filter Sets for Fluorescence Wavelength Discrimination

In order to facilitate the detection of two or more analytes in a single well of a standard multi-well plate reader, it was necessary to install filter sets on the plate reader that allowed fluorescence wavelength discrimination to occur with respect to peak emission, and the bandwidth of the filters was such that it restricted wavelength overlap between the peak fluorophore outputs.

Fluorescence measurements, either of fluorophores directly attached to the detection antibodies, or of fluorescent substrates of enzymes attached to the detection antibodies, were made in each well, with sequential or simultaneous excitation and emission being carried out, depending on the capabilities of the plate reader.

### Example 26

### Multiplex Kits

Duoplex kit: The following components are contained in a duoplex kit for measurement of two analytes, where the secondary antibodies are directly tagged with different fluorophores: Primary antibody to Target 1. Primary antibody to Target 2, Secondary antibody to Target 1. Secondary antibody to Target 2, Wash buffer, Cell Lysis buffer (if required), fluorescence enhancer solution (if required), Assay plate (pre-coated with immobilisation agent to immobilise Primary Antibodies).

Trioplex kit: The following components are contained in a trioplex kit for measurement of three analytes, where the secondary antibodies are directly tagged with different fluorophores: Primary antibody to Target 1, Primary antibody to Target 2, Primary antibody to Target 3, Secondary antibody to Target 1, Secondary antibody to Target 2, Secondary antibody to Target 3, Wash buffer, Cell Lysis buffer (if required), fluorescence enhancer solution (if required), Assay plate (pre-coated with immobilisation agent to immobilise Primary Antibodies).

### Example 27

### Sequential Multiplex Analysis

Cells are lysed with a buffer containing Triton X-100, buffer, and other components including phosphatase inhibitors, including sodium vanadate and sodium pyrophosphate. After addition of the lysis buffer, the plates are mixed gently, and then a sample (20 uL) taken from each well and placed into a 96-well assay plate.

The assay plate has wells pre-coated an immobilisation agent, in this case streptavidin. To the assay plate is then added three pairs of antibodies, being capture and detection antibodies for detection of three different cellular analytes. The primary (capture) antibody of each pair is, in this example, labelled with biotin to facilitate capture by the immobilisation agent on the assay wells. Suitable concentrations of the primary (capture) and secondary (detection) antibodies are each 50 ng/mL, for each pair of antibodies. The total binding capacity of the streptavidin in each well is approximately 2 ug.

The secondary (detection) antibodies of each of the three pairs of antibodies are labelled with 3 different enzymes, to allow reaction with the subsequently added enzyme substrates that would produce products with specific fluorescence characteristics. Three suitable enzymes are horseradish peroxidase, alkaline phosphatase, and beta-galactosidase.

For example, antibodies specifically selected to measure three intracellular targets, such as p-ERK, p-AKT and p-CREB may be used. The p-ERK assay secondary (detection) antibody was labelled with beta-galactosidase, the p-AKT secondary (detection) antibody with alkaline phosphatase (AP), and the p-CREB secondary (detection) antibody with horseradish peroxidise (HRP).

After incubation for 1 hour with gentle mixing at optimal speed of 300 rpm, the wells are washed with wash buffer, and a solution containing two combined enzyme substrates added to each well to measure the HRP and AP levels, which gives a measure of p-CREB and p-AKT, respectively. These substrates are: (a) 4-Methylumbelliferyl phosphate, being the substrate for alkaline phosphatise; and (b) 10-Acetyl-3,7-dihydroxyphenoxazine (ADHP), being the substrate for horseradish peroxidase. Concentrations of these substrates may be optimised and pre-determined for the assay kit. The buffer utilised for these reactions is also optimised and pre-determined for the assay kit.

After incubation for 15 minutes with substrate solution, the plates are read in a plate reader to measure conversion of substrates to specific fluorescence products, the wavelengths of excitation and emission being shown below. These were assayed in a plate reader with capability to measure the necessary specific wavelengths following excitation at specific wavelengths.

After reading the wells for these two analytes, the wells are washed with buffer to remove the enzyme substrates. After this wash step, a solution containing a single enzyme substrate is added to each well to measure the beta-galactosidase levels, being a reflection of the levels of p-ERK in each well. This substrate is Fluorescein di-beta-D-galactopyranoside (FDG), being a specific substrate for beta-galactosidase. After incubation for 15 minutes with this substrate solution, the plates are read a second time in a plate reader for conversion of this substrate to specific fluorescence product, the wavelengths of excitation and emission as shown below. These are assayed in a plate reader with capability to measure the necessary specific wavelengths following excitation at specific wavelengths.

Wavelengths (excitation/emission):

Wavelengths: 360/450, Enzyme: Alkaline Phosphatase

Wavelengths: 460/520, Enzyme: Beta-galactosidase

Wavelengths: 550/610, Enzyme: Horseradish Peroxidase

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The subject headings used herein are included only for the ease of reference of the reader and should not be used to limit the subject matter found throughout the disclosure or the claims. The subject headings should not be used in construing the scope of the claims or the claim limitations.

1. A method for detecting a non-nucleic acid analyte in a sample, the method comprising:
providing a reaction vessel;
providing a solid substrate comprising a bound immobilisation agent;
providing an antibody capture agent in solution which can bind the analyte, wherein the capture agent comprises a ligand for the immobilisation agent;
providing a detectable agent in solution which can bind to the analyte;
contacting the sample, the capture agent, the detectable agent and the solid substrate in the reaction vessel;
washing the solid substrate in the reaction vessel to remove the capture agent and the detectable agent not bound to the solid substrate via the ligand; and
detecting the analyte by detecting the presence of the detectable agent bound to the solid substrate in the reaction vessel.

2. The method according to claim 1 wherein the immobilisation agent and the ligand comprise a binding pair comprising an anti peptide tag antibody and a peptide tag.

3. The method according to claim 2, wherein the peptide tag comprises the amino acid sequence DYKDDDDK (SEQ ID NO. 1).

4. The method according to claim 1, wherein the antibody capture agent comprises a plurality of ligands.

5. The method according to claim 1, wherein the concentration of the capture agent is in a range selected from 10 to 1000 ng/ml or 50 to 500 ng/ml.

6. The method according to claim 1, wherein the detectable agent comprises an antibody.

7. The method according to claim 1, wherein the detectable agent comprises a detectable tag.

8. The method according to claim 7, wherein the detectable tag comprises an enzyme, a fluorophore, or a lanthanide.

9. The method according to claim 1, wherein the sample and the solid substrate are contacted prior to contacting with the capture agent and/or the detectable agent.

10. The method according to claim 1, wherein the sample and the solid substrate are first contacted in the reaction vessel.

11. The method according to claim 1, wherein the sample and the solid substrate are not substantially incubated prior to contacting with the capture agent and/or the detectable agent.

12. The method according to claim 1, wherein the capture agent and the detectable agent are contacted prior to contacting with either or both of the sample and the solid substrate.

13. The method according to claim 12, wherein the capture agent and the detectable agent are contacted in a separate vessel.

14. The method according to claim 1, wherein there is an incubation of the contacted sample, the solid substrate, the capture agent and the detectable agent prior to washing of the solid substrate.

15. The method according to claim 14, wherein the incubation is 2 hours or less.

16. The method according to claim 14, wherein the incubation is 30 minutes or less.

17. The method according to claim 1, wherein the contacting comprises providing the capture agent and the sample to the reaction vessel, washing the solid substrate and subsequently providing the detectable agent to the reaction vessel.

18. The method according to claim 1, wherein the reaction vessel comprises the solid substrate.

19. The method according to claim 1, wherein a plurality of different analytes is detected in the reaction vessel.

20. The method according to claim 1, wherein the immobilisation agent and the ligand comprise a binding pair comprising avidin, streptavidin and/or a derivative thereof and biotin and/or a derivative thereof.

21-47. (canceled)


SEQUENCE LISTING (7 sequences)

SEQ ID NO: 1 - protein, 8 aa, Artificial Sequence
  FLAG peptide
DYKDDDDK

SEQ ID NO: 2 - protein, 23 aa, Homo sapiens
KRITVEEALAHPYLEQYYDPTDE

SEQ ID NO: 3 - protein, 6 aa, Artificial Sequence
  Polyhistidine tag
HHHHHH

SEQ ID NO: 4 - protein, 10 aa, Homo sapiens
EQKLISEEDL

SEQ ID NO: 5 - protein, 9 aa, Influenza A virus
YPYDVPDYA

SEQ ID NO: 6 - protein, 11 aa, Vesicular stomatitis virus
YTDIEMNRLGK

SEQ ID NO: 7 - protein, 11 aa, Herpes Simplex Type 1
QPELAPEDPED